(19) United States
(12) Patent Application Publication
(10) Pub. No.: US 2025/0387312 A1
(43) Pub. Date: Dec. 25, 2025

(54) COSMETIC FORMULATIONS COMPRISING A BIOPOLYMER

(71) Applicant: 11584022 CANADA INC., Montreal, Quebec (CA)

(72) Inventors: Thomas DI NARDO, Montreal (CA); George DECKNER, Cincinnati, OH (US)

(73) Assignee: 11584022 CANADA INC., Montreal, Quebec (CA)

(21) Appl. No.: 18/879,362

(22) PCT Filed: Jun. 29, 2023
(86) PCT No.: PCT/IB2023/056723

§ 371 Date: Dec. 27, 2024

Related U.S. Application Data
(60) Provisional application No. 63/356,665, filed on Jun. 29, 2022.

(51) Int. Cl. A61K 8/65; A61K 8/04; A61K 8/64; A61Q 17/04; A61Q 19/00

(52) U.S. Cl. CPC A61K 8/65; A61K 8/044; A61K 8/64; A61Q 17/04; A61Q 19/00; A61K 2800/10

(57) ABSTRACT

Described herein are cosmetic compositions comprising biopolymer molecules that have been mechanically processed into a stable homogeneous aqueous suspension having insoluble and/or semi-soluble biopolymer particles stably dispersed within a polar solvent. The cosmetic compositions may be formulated as a sunscreen composition, a skin care composition, and a hair care composition. In embodiments the biopolymer is selected from chitin, chitosan, cellulose, hemicellulose, lignin, amylose, actin, fibrin, collagen, silk, fibroin, keratin, wool, alginic acid and mixtures thereof. In embodiments the biopolymer(s) has been mechanically processed under high-shearing conditions and/or high mechanical energy to obtain a paste, ointment, cream, lotion, gel or milk.

### CROSS REFERENCE TO RELATED APPLICATION

The present application claims priority to provisional patent application U.S. 63/356,665 (filed Jun. 29, 2022), the content of which is incorporated herein in its entirety.

### FIELD OF THE INVENTION

The invention relates to the field of cosmetics, and more particularly to new formulations comprising homogeneous suspensions of biopolymer(s) for use as sunscreen, skin care and/or hair care.

### BACKGROUND OF THE INVENTION

The cosmetic industry is permanently searching for new natural, biocompatible, biodegradable and non-toxic ingredients. For instance, microplastics are commonly used in the cosmetic industry but there are not many environmentally friendly alternatives yet.

Natural polymers, or biopolymers, are polymers that are abundant, natural and renewable, making them an attractive resource for the cosmetic industry. However, most abundant biopolymers such as cellulose and chitin are insoluble, thereby limiting and complicating their use. Advantageously, Applicant has described in WO 2022/137184 new homogeneous suspensions of biopolymers that are homogeneous and stable that may find numerous applications in the cosmetic industry.

Accordingly, there is still a need for a biodegradable alternative to replace microplastics in cosmetic products.

Also, there is still a need for cosmetic formulations, particularly sunscreen, skin care and hair care formulations, comprising natural, biocompatible, biodegradable and non-toxic ingredients such as cellulose, chitin and the like.

The present invention addresses these needs and other needs as it will be apparent from the review of the disclosure and description of the features of the invention hereinafter.

### BRIEF SUMMARY OF THE INVENTION

According to one aspect, the invention relates to a cosmetic composition comprising biopolymer molecules that have been mechanically processed into a stable homogeneous aqueous suspension, and wherein the stable homogeneous aqueous suspension comprises insoluble and/or semi-soluble biopolymer particles stably dispersed within a polar solvent.

According to another aspect, the invention relates to a sunscreen composition comprising: a biopolymer and at least one of a sun protection factor (SPF) booster, a sensory enhancer, a film former, an emulsifier, a co-emulsifier, an emollient, a UVB filter, a UVA filter, and a broad-spectrum filter.

According to another aspect, the invention relates to the use of a sunscreen composition as defined herein for protecting the skin against the sun.

According to another aspect, the invention relates to a method of skin protection, comprising applying onto the skin a sunscreen composition as defined herein.

According to another aspect, the invention relates to a skin care composition comprising: a biopolymer and at least one of a humectant, an emollient, an occlusive, a ceramide, an emulsifier, a thickening agent, a preservative, an antioxidant, a fragrance, and a functional ingredient.

According to another aspect, the invention relates to the use of a skin care composition as defined herein for benefiting the skin, e.g., moisturizing the skin, improving skin appearance, protecting from environmental damages, fighting the effects of aging, removing wrinkles and/or sunspots, etc.

According to another aspect, the invention relates to a method of protecting the skin and/or improving appearance of the skin, comprising applying onto the skin a skin care composition as defined herein.

According to another aspect, the invention relates to a hair care composition comprising: a biopolymer and at least one of a surfactant, a conditioning surfactant, a cosurfactant, conditioning emollient, a thickening agent, an opacifier, a ceramide, a conditioner, a pH buffer, a chelating agent, a preservative, an emulsifier, a stabilizer, an antioxidant, a fragrance, and a functional ingredient.

According to another aspect, the invention relates to the use of a biopolymer for replacing microplastics in a cosmetic composition, said comprising substituting at least a portion of said microplastics with a biopolymer suspension, the biopolymer suspension comprising a suspension of insoluble and/or semi-soluble particles stably dispersed within a polar solvent.

According to another aspect, the invention relates to a method for replacing microplastics in a sunscreen composition, comprising: (1) providing a biopolymer suspension comprising a suspension of insoluble and/or semi-soluble particles stably dispersed within a polar solvent; and (2) mixing the biopolymer suspension with at least one of a sensory enhancer, a film former, an emulsifier, an emollient, a UVB filter, a UVA filter, and a broad-spectrum filter.

According to another aspect, the invention relates to a method for replacing microplastics in a skin care composition, comprising: (1) providing a biopolymer suspension comprising a suspension of insoluble and/or semi-soluble particles stably dispersed within a polar solvent; and (2) mixing the biopolymer suspension with at least one of a humectant, an emollient, an occlusive, a ceramide, an emulsifier, a thickening agent, a preservative, an antioxidant, a fragrance, and a functional ingredient.

According to another aspect, the invention relates to a method for replacing microplastics in a hair care composition, comprising: (1) providing a biopolymer suspension comprising a suspension of insoluble and/or semi-soluble particles stably dispersed within a polar solvent; and (2) mixing the biopolymer suspension with at least one of a surfactant, a conditioning surfactant, a cosurfactant, conditioning emollient, a thickening agent, an opacifier, a ceramide, a conditioner, a pH buffer, a chelating agent, a preservative, an emulsifier, a stabilizer, an antioxidant, a fragrance, and a functional ingredient.

Additional aspects, advantages and features of the present invention will become more apparent upon reading of the following non-restrictive description of preferred embodiments which are exemplary and should not be interpreted as limiting the scope of the invention.

### DETAILED DESCRIPTION OF EMBODIMENTS

In the following description of the embodiments are illustrations of examples by which the invention may be practised. It will be understood that other embodiments may be made without departing from the scope of the invention disclosed. Unless defined otherwise, all technical and scientific terms used herein have the same meaning as commonly understood by one of ordinary skill in the art to which the invention belongs.

### General Overview

In PCT publication WO 2022/137184 entitled “HOMOGENEOUS BIOPOLYMER SUSPENSIONS, PROCESSES FOR MAKING SAME AND USES THEREOF” (the content of which is incorporated herein by reference in its entirety), Applicant has described the preparation of stable homogeneous suspensions of insoluble and/or semi-soluble biopolymers.

The present invention generally relates to the uses of such homogeneous suspensions of biopolymers in the manufacture of “cosmetic composition(s)” or “cosmetic formulation(s)” (both terms are used herein interchangeably). Particular aspects concern new sunscreen, skin care and hair care formulations/compositions and processes for obtaining the same. The present invention encompasses, but is not limited to, general and specific compositions described in WO 2022/137184.

### Biopolymer

The cosmetic compositions in accordance with the present invention (e.g. sunscreen, skin care and/or hair care compositions) minimally require a biopolymer. As used herein, the term “biopolymer” refers to natural polymers produced by the cells of living organisms. Biopolymers consist of monomeric units that are covalently bonded to form larger molecules. The present invention encompasses polypeptides, polysaccharides and polynucleotides biopolymers. Other examples of biopolymers include natural rubbers (polymers of isoprene), suberin and lignin (complex polyphenolic polymers), cutin and cutan (complex polymers of long-chain fatty acids) and melanin. In embodiments the biopolymers used as starting materials and obtained in the suspensions are substantially pure, i.e., they consist of only purified natural polymers.

Preferably, the biopolymer used in the cosmetic formulation is substantially free from chemical residues and any of such chemical residue is absent or present in undetectable or trace amounts. Preferably, the biopolymer is substantially free from chemical residues and any of such chemical residue is absent or present in undetectable or trace amounts. As used herein, “substantially free from chemical residues” means that chemical compounds, such as acids, bases, reactive chemicals, organic salts and/or inorganic salts, surfactants, dispersing agents (e.g., Twin 80™), a silanizing reagent, acrylamide, etc. are totally absent or merely present in undetectable or trace amounts in the final composition or final suspension. In embodiments, the biopolymer(s) will constitute at least 98%, or at least 99% or at least 99.9% or at least 99.99% by weight of the organic compounds in the biopolymer composition or suspension, i.e., the biopolymer composition or suspension will contain less than 2% or less than 1%, less than 0.1%, or less than 0.01%, or less than 0.001% by weight of organic components other than the biopolymer(s) or degradation product(s).

In accordance with the present invention, the biopolymer may be insoluble or semi-soluble in water. As used herein, the term “insoluble biopolymer” refers to a biopolymer that is “insoluble” in a polar solvent (particularly water) and this term encompasses equivalent terms such as “non-water-soluble”, or “not soluble in water”, or “water-insoluble” or “indissoluble”. Insolubility can typically be observed by a separation, i.e., two separate phases in an aqueous mixture, for instance biopolymer deposits/sediments at a bottom or floating at the top of the aqueous mixture. In accordance with the present invention, examples of insoluble biopolymers include, but are not limited to, chitin, chitosan, cellulose, hemicellulose, lignin, amylose, actin, fibrin, collagen, silk, fibroin, keratin, wool, alginic acid and mixtures thereof. As used herein, the term “semi-soluble biopolymer” refers to a biopolymer that may be solubilized in a polar solvent such as water, but under certain conditions (e.g., molecular weight, heat, addition of chemicals such as acids, alcohols, surfactants, etc.). In accordance with the present invention, examples of semi-soluble biopolymers include, but are not limited to gelatin, pectin, starch, amylopectin, agarose, hyaluronic acid, RNA, DNA, xanthan gum, latex, polymannans, suberin, cutin, cutan, and mixtures thereof.

In embodiments the insoluble biopolymer is selected from chitin, chitosan, cellulose, hemicellulose, lignin, amylose, actin, fibrin, collagen, silk, fibroin, keratin, wool, and mixtures thereof. In embodiments the semi-soluble biopolymer is selected from gelatin, pectin, starch, amylopectin, agarose, alginic acid, alginate, hyaluronic acid, RNA, DNA, xanthan gum, guar gum, carrageenan, latex, polymannans, suberin, cutin, cutan, and mixtures thereof.

Various sources of biopolymers may be used and the present invention is not limited to particular sources of materials. For instance, suitable sources of chitin may include, but are not limited to, green plants, algae, and fungi. Suitable sources of chitin and chitosan may include, but are limited to, fungi, crustaceans (e.g. crabs and shrimps) and insects. In embodiments the insoluble or semi-soluble biopolymer is obtained from fungi and mushrooms. In embodiments the insoluble or semi-soluble biopolymer is obtained from plant materials including, but not limited to, roots, tubers, leaves, petals, seeds, fruits, etc. Preferred sources are non-animal sources.

In embodiments the biopolymer is a 100% natural biopolymer such as SunSpheres Bio™ (microcrystalline cellulose, Dow Chemical), Chemjac™ (amorphophallus konjac root extract and xanthan gum, Chemspire), Kelset™ (sodium alginate, Dupont), Instant Pure-Flo F™ (corn starch, Ingredion), Gelcarin™ GP 379 (carrageenan, iota form, Dupont), Betafib™ MCF (cellulose (and) water-microfibrillated cellulose, Cosun Biobased Products), Betafib™ ETD (cellulose and cellulose gum-microfibrillated cellulose, Cosun Biobased Products), Exilva™ FM02-V,L (cellulose-microfibrillated cellulose, Borregard), Naturesoft™ 800 (cellulose-micro powders), Kelcogel™ CG-HA (gellan gum-CP, Kelco), agar agar, and agarose, mushroom chitosan (e.g., GBS003, Qingdao Chibio Biotech), fungal chitosan (e.g., GBS010, Qingdao Chibio Biotech; or from Kraeber & Co), a fungal derived chitosan (e.g., Kiosmetine-CS™, Kitozyme).

In embodiments the biopolymer is a natural derived biopolymer such as Natrathix™ bio cellulose (cellulose gum, Ashland), Aquasorb™ A500 (cellulose gum, Ashland), Polysurf™ CS 67/Natrosol™ CS plus 330 (cetyl hydroxyethylcellulose, Ashland), Structure XL™ (hydroxy propyl starch phosphate, Nouryon), CD-58 (chitosan succinimide, Onlystar Bio-Technology Ltd), carboxymethyl chitosan derivative (GBS010, Qingdao Chibio Biotech), Makimousse™ 7/400 (sodium polyacrylate starch-kobo products, Daito Kasel Kogyo), Salanjul/Sanfresh™ 1000/300sp (sodium polyacrylate starch, Iwase Cosfa USA Inc./Sanyo), Antaron ECoT ethylcellulose (ethyl cellulose, Ashland).

In embodiments the biopolymer comprises a synergistic biopolymer combination, such as Chemjac™ (amorphophallus konjac root extract and xanthan gum, Chemspire), PemuPur™ start (microcrystalline cellulose (and) sphingomonas ferment extract (and) cellulose gum, Lubrizol), Nomcort CG (xanthan gum, Ceratonia siliqua gum, Ikeda).

The biopolymer may also comprise other biopolymer actives such as B-CAN™ 55% (oat beta glucan, Adams Food Ingredients) and/or mushroom derived beta glucan.

The present invention encompasses mixtures of two, three, four, five or more insoluble biopolymers including, but not limited to, chitin+chitosan, chitin+cellulose, chitin+collagen, chitin+silk, chitosan+silk, chitosan+cellulose, chitosan+collagen, cellulose+collagen, cellulose+silk, collagen+silk, etc. The present invention also encompasses mixtures of two, three, four, five or more semi-soluble biopolymers including, but not limited to agarose+DNA, xanthan gum+starch, latex+alginate, xanthan gum+DNA, guar gum+cutan, etc. It may also be envisioned to mix together two, three, four, five or more insoluble and semi-soluble biopolymers including but not limited to chitin+agarose, chitosan+agarose, chitin+gelatin, chitin+xanthan gum, chitosan+xanthan gum, chitin+sodium hyaluronate, chitosan+sodium hyaluronate, cellulose+sodium hyaluronate, chitin+agarose, chitosan+agarose, cellulose+agarose.

The present invention encompasses also combinations of hydrophobically modified biopolymers and unmodified biopolymers which can form stable, viscous oil in water emulsions when dispersed using high shear processes and/or mechanical energy (with or without emulsifiers), Examples of hydrophobically modified biopolymers include Natrosol™ CS Plus 330/Polysurf™ CS 67 (cetyl hydroxyethylcellulose, Ashland), StarDesign Ultra™ (sodium starch octenylsuccinate, Cargill Beauty), Inutec™ SP1 (inulin lauryl carbamate, Beneo), Texturlux Stabil™ (hydrolyzed corn starch hydroxyethyl ether, Primient).

In embodiments, the biopolymer consists of a biopolymer compositions comprising biopolymer molecules that have been mechanically processed into a stable homogeneous aqueous biopolymer suspension. As used herein the term “homogeneous” generally refers to the appearance of the suspension under the naked eye (e.g., uniform color, uniform texture, etc.). Homogenous as used herein does not exclude the possibility that the suspension is “heterogenous” at the molecular level (e.g., various particles size, presence of aggregates, etc.). As used herein, the terms “stable homogeneous aqueous biopolymer suspension”, or similar terms that may be used herein interchangeably such as “homogenous biopolymer suspension” or “stable biopolymer suspension” or simply “biopolymer suspension”, all refer to a suspension of insoluble and/or semi-soluble biopolymer particles that have been stably dispersed within a polar solvent. The polar solvent may be a polar protic solvent or a polar aprotic solvent. The polar solvent may be an aqueous solvent. The insoluble and/or semi-soluble biopolymer particles that are present in biopolymer suspensions may be shaped like fibers and/or like agglomerated spheres or agglomerated bodies. Stability of the biopolymer suspensions may be assessed by any suitable means. In preferred embodiments, the stability is measured or observed by a lack of separation, i.e., one single phase instead of two separate phases in an aqueous mixture, for instance absence of biopolymer deposits/sediments at a bottom or floating at the top of the aqueous mixture. Preferably, biopolymer suspensions in accordance with the present invention are stable (e.g., absence of separation) for at least 1 day, or at least 1 week, or at least one month, or at least one year or more.

Notwithstanding the above, those skilled in the art understand that insoluble and/or semi-soluble biopolymers may never become truly soluble. Instead, they become “swellable” and bind water which is why the biopolymer becomes a viscous suspension during the high-shearing conditions and/or mechanical energy to which the biopolymer(s) are submitted in accordance with the present invention. Accordingly, the present invention encompasses both, “swellable biopolymers” as well as “non-swellable biopolymers” since a non swellable polymer could be swellable using high-shearing and/or mechanical energy. As used herein, swellable biopolymers encompasses biopolymers that absorb and bind water, which results in an increase in their particle size and water dispersion viscosity.

In embodiments, the biopolymer is swellable with wet ball milling. This may include, but it is not limited to, chitin, chitosan, hemicellulose and pregelatinized corn starch.

In embodiments, the biopolymer is swellable with high shear processes other than ball milling. This may include, but it is not limited to, microcrystalline cellulose, microfibrillated cellulose, nano cellulose, hairy nanocellulose, konjac glucomannan, hydroxypropyl starch phosphate, high acyl gellan gum, gellan gum, carboxymethyl starch, carboxymethyl cellulose (low ds type), agar agar, and agarose.

It is also conceivable in accordance with the present invention to use soluble biopolymers including, but not limited to, xanthan gum, diutan gum, sodium alginate, sclerotium gum.

In embodiments, the biopolymer molecules or particles that are part of the cosmetic composition have been mechanically processed into a stable homogeneous aqueous biopolymer suspension. In embodiments the mechanical processing involves high-shearing conditions and/or high mechanical energy. In embodiments the high-shearing conditions and/or high mechanical energy is obtained by a process including, but not limited to mechanical shearing, shear thinning, planetary ball milling, rolling mill, vibrating ball mill, tumbling stirred ball mill, horizontal media mill, colloid milling. As indicated hereinafter, the high-shearing conditions and/or high mechanical energy can be carried out for a duration, under parameters, under suitable conditions, etc. until a desirable change of state is obtained, e.g., change of color, a change in viscosity, a change from a slurry to a paste, ointment, cream, lotion, gel or milk, etc.

Without wishing to be bound by theory, submitting the biopolymer to high-shearing conditions and/or high mechanical energy improves biopolymer performance not seen using conventional processes, including improved rheological properties such as viscosity, shear thinning properties, and high yield value. Biopolymers dispersed using high shear processes with a fatty alcohol may contain lamellar crystalline gel networks (LGN) that may synergistically increase the viscosity of the biopolymer dispersions. Oil+water biopolymer dispersions in accordance with the present invention may also contain Pickering emulsion wherein the water-in-oil or oil-in-water emulsion is stabilized by the biopolymer.

In embodiments the high-shearing conditions and/or high mechanical energy requires using a suitable device or apparatus including, but not limited to, ball miller (e.g., planetary ball miller, rolling miller, vibrating ball miller, tumbling stirred ball miller, horizontal media mill, colloid miller, a magnetic miller), a twin-screw extruder, a high-pressure homogenizer, a blade homogenizer, a stirring homogenizer, a disperser, a rotor-stator homogenizer, a high-shear mixer, a plowshare mixer, a dynamic mixer, a plough mixer, a turbine mixer, a speed mixer, an attrition miller, a sonicator (e.g. high shear ultrasonic processes), a tissue tearor, a cell lysor, a polytron, a ribbon agitator, a microfluidizer, a high pressure homogenizer, and combinations thereof. In preferred embodiments, the present invention utilizes ball milling under wet conditions. Particular examples of ball millers include, but are not limited to, vertical planetary mill (e.g., Tencan XQM-2A™) with 100 ml capacity zirconia jars and 10 mm diameter zirconia balls, Flacktek™ speed mixer (DAC 330-11 SE) with 40 mL zirconia jar with 5 mm diameter zirconia balls or zirconia rings, 1.5 L Supermill Plus™ with 1.4-1.7 mm zirconia beads and Netzsch Labstar with 0.6-0.8 mm beads or 1.4-1.7 mm beads.

In particular embodiments, biopolymer compositions and suspensions in accordance with the present invention are obtained using a particular protocol referred herein as the “10+1 Alt method”. This method comprises milling of the biopolymer for a certain period of time (e.g., 10 min) followed by a short pause (e.g., 1 min) then milling in the opposite direction for a certain period of time (e.g., 10 min) for a total of 1 hour, or 2 hours, or 3 hours, or 5 hours, 10 hours, or 12 hours.

Advantageously, the viscosity of the compositions/suspensions can be altered by varying the high-shearing conditions and/or mechanical energy to which the biopolymer(s) are submitted. These conditions can be adjusted to obtain a stable homogeneous suspension (e.g., a stable colloidal homogeneous suspension) having a desired viscosity. Typically, providing more mechanical energy will increase the shearing and will reduce accordingly the viscosity of the end product. The biopolymer itself and/or the final compositions for drug delivery may be formulated as a paste, an ointment, a cream, a lotion, a gel or a milk.

In embodiments, the biopolymer, and/or the biopolymer compositions comprising biopolymer molecules or particles is a colloidal homogeneous biopolymer suspension. In embodiments, the colloidal homogeneous suspension comprises colloids having a range from about 1 nm to about 1 μm.

In embodiments, the biopolymer, and/or the biopolymer compositions comprising biopolymer molecules, comprises biopolymer fibers. In embodiments the fibers have of a width of about 1 nm to about 5 μm, or about 5 nm to about 5 μm, about 7 nm to about 5 μm, or about 10 nm to about 5 μm, or about 20 nm to about 5 μm, or about 25 nm to about 5 μm, or about 30 nm to about 5 μm, or about 35 nm to about 5 μm, or about 35 nm to about 3 μm. In embodiments the fibers having of a width of at least 1 nm, or at least 5 nm, or at least 10 nm, or at least 20 nm, or at least 30 nm, or at least 40 nm, or at least 50 nm, or at least 75 nm, or at least 100 nm, or at least 250 nm, or at least 500 nm, or at least 750 nm, or at least 1 μm, or at least 2 μm, or at least 3 μm, or at least 4 μm, or at least 5 μm, or wider.

In embodiments the biopolymer, and/or the biopolymer compositions comprising biopolymer molecules, comprises biopolymer fibers having a length of about 1 nm to about 200 μm, of about 10 nm to about 100 μm, or about 50 nm to about 10 μm, or about 100 nm to about 10 μm, or about 500 nm to about 10 μm, or about 750 nm to about 10 μm, or about 800 nm to about 10 μm, or about 900 nm to about 5 μm, or about 1 μm to about 10 μm, or about 1 μm to about 5 μm, or about 1 μm to about 3 μm. In embodiments the fibers have of a length of at least 1 nm, or at least 10 nm, at least 50 nm, or at least 100 nm, or at least 250 nm or at least 500 nm, or at least 750 nm, or at least 800 nm, or at least about 900 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or longer. In embodiments, a dry particle size range may be between about 1 nm to about 1 μm, or up to 10 μm, and a wet particle size range may be between about 200 nm to about 20 μm, or up to 200 μm.

In embodiments the biopolymer, and/or the biopolymer compositions comprising biopolymer molecules, comprises biopolymer fibers having both: (i) a width greater than 20 nm (e.g., at least 25 nm, or at least 40 nm, or at least 50 nm,) and a length greater than 50 nm (e.g., at least 100 nm, or at least 500 nm, or at least 1 μm, or at least 2 μm); or (ii) a width greater than 32 nm (e.g., at least 35 nm, or at least 40 nm, or least 50 nm) and a length of than 50 nm (e.g., at least 100 nm, or at least 500 nm, or at least 1 μm, or at least 2 μm); or (iii) a width greater than 20 nm (e.g., at least 25 nm, or at least 40 nm, or least 50 nm) and a length of than 500 nm (e.g., at least 600 nm, or at least 750 nm, or at least 1 μm, or at least 2 μm); or (iv) a width greater than 30 nm (e.g., at least 35 nm, or at least 40 nm, or least 50 nm) and a length of than 800 nm (e.g., at least 900 nm, or at least 1 μm, or at least 2 μm); or (v) a width greater than 8 nm (e.g., at least 10 nm, at least 25 nm, or at least 35 nm, or at least 40 nm, or least 50 nm) and a length of than 340 nm (e.g., at least 350 nm, or at least 500 nm, at least 750 nm, or at least 900 nm, or at least 1 μm, or at least 2 μm); or (vi) a width greater than 11 nm (e.g., at least 15 nm, at least 25 nm, or at least 35 nm, or at least 40 nm, or least 50 nm) and a length of than 166 nm (e.g., at least 200 nm, or at least 350 nm, or at least 500 nm, at least 750 nm, or at least 900 nm, or at least 1 μm, or at least 2 μm); or (viii) a width greater than 32 nm (e.g., at least 35 nm, or at least 40 nm, or least 50 nm) and a length greater than 800 nm (e.g., at least 900 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or longer).

In embodiments the biopolymer, and/or the biopolymer compositions comprising biopolymer molecules, comprises biopolymer fibers wherein the average width and average length of the fibers in the composition are as defined hereinabove, e.g. an average width greater than 20 nm (e.g., at least 25 nm, or at least 40 nm, or at least 50 nm) and an average length greater than 50 nm (e.g., at least 60 nm, at least 75 nm, or at least 100 nm, or at least 500 nm, at least 750 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or wider).

In embodiments the biopolymer suspensions and/or biopolymer compositions comprising biopolymer molecules, have a pH between about 6.5 and about 8.5. In particular embodiments the biopolymer suspension is a chitosan suspension having a pH between about 7.8 and about 8.1.

In embodiments the biopolymer, and/or the biopolymer compositions comprising biopolymer molecules, comprises biopolymer fibers having both a crystalline region and an amorphous region. In embodiments the stable homogeneous suspension comprises biopolymer fibers having a globular shape. In embodiments the stable homogeneous suspension is comprised of mainly, or only, of suspended biopolymer nanofibrils.

Those skilled in the art are aware that particle size measurements may vary according to the measurement method and the state of the particles (e.g., particles in a wet state are typically larger than the same particles in a dry state). Typically, the particles will be in a wet or suspended stage when measured by dynamic light scattering (DLS) and in a dry stage when measured by scanning electron microscopy (SEM).

In embodiments the biopolymer, and/or the biopolymer compositions comprising biopolymer molecules, comprises agglomerated spheres of alginic acid having an average size of about 40 nm to about 80 nm, or about 45 nm to about 75 nm, as measured by scanning electron microscopy (SEM). In embodiments, the stable homogeneous suspension comprises agglomerated spheres of alginic acid having a median size of about 30 nm to about 70 nm or about 35 nm to about 65 nm, average size of about 40 nm to about 80 nm, or about 45 nm to about 75 nm, as measured by scanning electron microscopy (SEM).

In embodiments the biopolymer, and/or the biopolymer compositions comprising biopolymer molecules, comprises agglomerated spheres of cellulose having an average size of about 50 nm to about 80 nm, or about 55 nm to about 75 nm, average size of about 40 nm to about 80 nm, or about 45 nm to about 75 nm, as measured by scanning electron microscopy (SEM). In embodiments the stable homogeneous biopolymer suspension comprises agglomerated spheres of cellulose having a median size of about 35 nm to about 75 nm or about 40 nm to about 65, average size of about 40 nm to about 80 nm, or about 45 nm to about 75 nm, as measured by scanning electron microscopy (SEM).

In embodiments the biopolymer, and/or the biopolymer compositions comprising biopolymer molecules, comprises agglomerated spheres of chitin having an average size of about 45 nm to about 85 nm, or about 50 nm to about 80 nm. In embodiments the stable homogeneous biopolymer suspension comprises agglomerated spheres of cellulose having a median size of about 45 nm to about 80 nm or about 50 nm to about 75 nm, as measured by scanning electron microscopy (SEM).

In embodiments the biopolymer, and/or the biopolymer compositions comprising biopolymer molecules, comprises agglomerated spheres of chitosan having an average size of about 75 nm to about 120 nm, or about 80 nm to about 115 nm, or about 85 nm to about 110 nm, as measured by scanning electron microscopy (SEM). In embodiments the stable homogeneous suspension comprises agglomerated spheres of chitosan having a median size of about 70 nm to about 100 nm or about 75 nm to about 95 nm, as measured by scanning electron microscopy (SEM).

In embodiments the biopolymer, and/or the biopolymer compositions comprising biopolymer molecules, comprises agglomerated spheres of silk having an average size of about 40 nm to about 165 nm, or about 45 nm to about 160 nm, as measured by scanning electron microscopy (SEM). In embodiments the stable homogeneous biopolymer suspension comprises agglomerated spheres of silk having a median size of about 40 nm to about 150 nm or about 45 nm to about 140, as measured by scanning electron microscopy (SEM).

In embodiments the biopolymer, and/or the biopolymer compositions comprising biopolymer molecules, comprises particles of one or more of alginic acid, cellulose, chitin, chitosan and silk, wherein the range of particle sizes, as measured by SEM is as defined in the tables and figures of WO 2022/137184.

In embodiments the biopolymer, and/or the biopolymer compositions comprising biopolymer molecules, is(are) characterized by visual properties like those depicted in the SEM images shown in the figures of WO 2022/137184.

In embodiments the biopolymer, and/or the biopolymer compositions comprising biopolymer molecules, is(are) characterized by a Fourier Transform Infrared Spectroscopy (FTIR) spectrum as depicted in the figures of WO 2022/137184.

In embodiments the biopolymer, and/or the biopolymer compositions comprising biopolymer molecules, is(are) characterized by Solid-State Nuclear Magnetic Resonance characterization (SSNMR) as depicted in the figures of WO 2022/137184.

In embodiments the biopolymer, and/or the biopolymer compositions comprising biopolymer molecules, is(are) characterized by Power X-Ray Diffraction (PXRD) pattern(s) as depicted in the figures of WO 2022/137184.

In embodiments the biopolymer, and/or the biopolymer compositions comprising biopolymer molecules, is(are) characterized by Dynamic Light Scattering (DLS) measurements like those reported in WO 2022/137184.

In embodiments the biopolymer, and/or the biopolymer compositions comprising biopolymer molecules, is(are) characterized by a transmittance spectrum as shown in the figures of WO 2022/137184.

In embodiments the biopolymer, and/or the biopolymer compositions comprising biopolymer molecules, is(are) characterized by a sweep suspension test as reported in WO 2022/137184.

In embodiments the biopolymer, and/or the biopolymer compositions comprising biopolymer molecules, is(are) characterized by a rheological behaviour as depicted in the figure(s) s of WO 2022/137184.

### Sunscreen Compositions

One particular aspect of the invention concerns sunscreen compositions comprising a biopolymer and/or a biopolymer suspension as described herein. In embodiments the biopolymer comprises, or alternatively consists of, a biopolymer composition comprising biopolymer molecules that have been mechanically processed into a stable homogeneous aqueous suspension (e.g., a suspension of insoluble and/or semi-soluble particles stably dispersed within a polar solvent).

Advantageously the sunscreen compositions may comprise smaller monomeric units of a multimeric biopolymer, such as Acetylglucosamine (GlcNAc) and/or oligomers of NAGs that may exhibit UV blocking properties.

In one embodiment, the sunscreen composition comprises a biopolymer and at least one of a sensory enhancer, a film former, an emulsifier, a coemulsifier, an emollient, a UVB filter, a UVA filter, a broad-spectrum filter and a preservative. In embodiments, the sunscreen composition comprises at least water, a biopolymer (acting as a thickener), a UVB absorber, a UVA absorber, an emollient, and a preservative. In embodiments, the sunscreen composition further comprises one or more of these optional ingredients: an humectant, an emulsifier and a film former.

In embodiments the sensory enhancer is selected from the group consisting of cyclopentasiloxane, cyclohexasiloxane, cyclopentasiloxane, cisiloxane, trisiloxane, dimethicone, caprylyl methicone, phenyltrimethicone, cetyl dimethicone, bis-hydroxyethoxypropyl dimethicone, dimethicone/vinyl dimethicone crosspolymer, dimethicone/dimethicone crosspolymer, PEG-20 sorbitan cocoate, phenoxyethanol, ethylhexylglycerin, potassium sorbate, phenoxyethanol, methylparaben, isopropylparaben, isobutylparaben, butylparaben and mixtures thereof.

In embodiments the film former is selected from the group consisting of cetyl dimethicone, stearyl dimethicone, phenyltrimethicone, bis-hydroxyethoxypropyl dimethicone, isododecane, polypropylsilsesquioxane, dimethicone/vinyl dimethicone crosspolymer, C30-45 alkyl methicone, C30-45 olefin, beeswax, Euphorbia cerifera (candelilla) wax, Copernicia prunifera (carnauba) wax, Isododecane, acrylates polytrimethylsiloxymethacrylate copolymer, hydrogenated polycyclopentadiene, galactoarabinan, methyl dihydroabietate, trimethylsiloxysilicate, trimethylsiloxysilicate, polypropylsilsesquioxane, C30-45 alkyldimethylsilyl polypropylsilsesquioxane, ethylenediamine/hydrogenated dimer dilinoleate copolymer bis-di-C14-18 alkyl amide, acrylates copolymer and mixtures thereof. Particular examples include, but are not limited to, Dermacryl E™ (styrene/acrylates copolymer (Nouryon)), Ganex™ V220 (Eicosene/VP), Cosmedia™ DC (hydrogenated dimer dilinoleyl/dimethylcarbonate (BASF)), Baycusan™ C 1000 (polyurethane-34 (Covestro), Hercolyn™ D (methyl hydrogenated rosinate (Pinova)), Tego™ SP 13-1, 13-6 (poly C10-30 alkyl acrylate (Evonik), Ganex™ P904 (butyl PVP (Ashland)).

In embodiments the emulsifier is selected from the group consisting of acrylates/C10-30 alkyl acrylate crosspolymer, potassium cetyl phosphate, sodium cetearyl sulfate, C20-22 alkyl phosphate, PEG-30 dipolyhydroxystearate, polyglyceryl-2 dipolyhydroxystearate, polyglyceryl-3 diisostearate and mixtures thereof. Particular examples include, but are not limited to, Emulsiphos™ (potassium cetyl phosphate+hydrogenated palm glyceride (Symrise)), Dracorin™ GOC (glyceryl oleate citrate+caprylic/capric triglyceride (Symrise)), Dracorin™ CE (glyceryl stearate citrate (Symrise)), polyglyceryl 10 stearate and triglyceryl stearate.

In embodiments the coemulsifier is selected from the group consisting of cetyl, stearyl alcohol, stearic acid, glyceryl stearate, sorbitan stearate, polyglyceryl 3 distearate and mixtures thereof. In embodiments the emollient is selected from the group consisting of C12-15 alkyl benzoate, trisiloxane, squalene, butyl octyl salicylate, isopropyl isostearate, isostearyl isostearate, caprylyl carbonate, isopropyl myristate, dibutyl adipate, caprylic/capric triglyceride, coco-caprylate, dicaprylyl carbonate, ethylhexyl methoxycrylene, diethylhexyl 2,6-naphthalate, butyloctyl salicylate, tridecyl salicylate, polyester-8, diethylhexyl syringylidene malonate, benzotriazolyl dodecyl p-cresol, propylheptyl caprylate and mixtures thereof.

In embodiments the UVB filter is selected from the group consisting of Uvivul™ T 150 (Ethylhexyl Triazone), titanium dioxide, benzophenone-3 (oxybenzone), octinoxate (ethylhexyl methoxycinnamate), octocrylene, ethylhexyl salicylate (octisalicylate), homosalate (homomenthyl salicylate), ensulizole (phenylbenzimidazole sulfonic acid), amiloxate (isoamyl p-methoxycinnamate), polysilicone-15, diethylhexyl butamido triazone (iscotrizinol), 4-methylbenzylidene camphor (enxacamene) and mixtures thereof.

In embodiments the UVA filter is selected from the group consisting of butyl methoxydibenzoylmethane, butyl methoxydibenzoylmethane (Avobenzone™), diethylamino hydroxybenzoyl hexyl benzoate (Univul™ a plus), terephthalylidene dicamphor sulfonic acid (Mexoryl™ SX), disodium phenyl dibenzimidazole tetrasulfonate (Neo Heliopan™ ap), Tinosorb™ S (bis-ethylhexyloxyphenol methoxyphenyl triazine), Tinosorb™ M (methylene bis-benzotriazolyl tetramethylbutylphenol (and) aqua (and) decyl glucoside (and) propylene glycol (and) xanthan gum), Tinosorb™ A2B (tris-biphenyl triazine (and) aqua (and) decyl glucoside (and) butylene glycol (and) disodium phosphate (and) xanthan gum) and mixtures thereof.

In embodiments the broad-spectrum filter is selected from the group consisting of bis-ethylhexyloxyphenol methoxyphenyl triazine, tris-biphenyl triazine (nano), methylene bis-benzotriazolyl tetramethylbutylphenol, drometrizole trisiloxane, zinc oxide and mixtures thereof.

In embodiments the SPF booster is selected from the group consisting of Soltex INO™ Polymer (acrylates/methacryloyloxvethyl phosphate copolymer, Dow Chemical), Soltex INO™ Polymer (acrylates/methacryloyloxvethyl phosphate copolymer, Dow Chemical), Solterra Boost™ (methyl cellulose, Dow Chemical), Synoxyl HSS™ (trimethoxybenzylidene pentanedione, Sytheon), SunSpheres™ Powder (styrene acrylates copolymer, Dow Chemical), SunSpheres BIO™ SPF Booster (microcrystalline cellulose, Dow Chemical), SunBoost ATB™ (Argania spinosa kernel oil (and) tocopheryl acetate (and) bisabolol, Kobo Products), NS Boost™ (3% active), NS Boost™ (mica, silica, Next Step Labs), LaraCare™ A200 (galactoarabinan, Lonza), HallBrite™ BHB (butyloctylsalicvlate, Hallstar), Dow Corning™ 5562 Carbinol Fluid (bis-hydroxyethoxypropyl dimethicone, Nexeo Solutions), Performacol™ 350 (C20-40 alcohols, New Phase Technologies), Corapan™ TQ (diethylhexyl 2,6-naphthalate, Symrise), Lexgard™ O (caprylyl glycol, Inolex), HallBrite™ BHB (butyloctylsalicylate, Hallstar), TDSA (tridecyl salicylate, Alzo Inc.), Karansun™ SC 50 concentrated (Pongamia seed oil, BioSynthis), Pongamia Butter (Pongamia extract, Biosynthis), Solastay™ S1 (ethylhexyl methoxycrylene, Hallstar), AvoBrite™ (acrylates copolymer, Hallstar), Tego™ SP 13-1, 13-6 (poly C10-30 alkyl acrylate, Evonik), Oleocraft™ HP 31, MP30, MP31 (polyamide 3, Croda), Koboguard™ HRPC (hydrogenated polycyclopentadiene (and) polyethylene (and) Copernicia cerifera (carnauba) wax (and) tocopherol, Kobo Products), Baycusan™ C1000 (polyurethane 34, 40% latex solids, Bayer Material Science), SolPerForm™ (water, hydrolyzed wheat protein/PVP crosspolymer, 29% solids, Croda) and mixtures thereof.

In embodiments the preservative is selected from the group consisting of benzyl alcohol, sodium benzoate, caprylyl glycol, glyceryl caprylate, phenoxyethanol, hexanediol, hydroxyacetophenone, sodium dehydroacetic acid, sodium phytate and mixtures thereof.

In embodiments the biopolymer plays the role of a thickening agent. Nevertheless, it may be envisioned to add thickening agent(s) to the sunscreen composition including, but not limited to cetyl alcohol, stearyl alcohol, carnauba wax, and stearic acid, hydroxyethylcellulose, locust bean gum, xanthan gum, gelatin, silica, bentonite, and magnesium aluminum silicate, carbomer, sodium chloride.

In embodiments the sunscreen composition comprises the following ingredients:

| | % (w/w) IN THE |
|---|---|
| FUNCTION(S) | FORMULATION |
| biopolymer | 0.3-10 |
| (dry weight) | |
| thickening agent | 0-5 |
| sensory enhancer | 1-3 |
| Humectant | 0-3 |
| film former | 0-3 |
| emulsifier | 0-5 |
| emollient | 10-20 |
| UVB filter | 0-20 |
| UVA filter | 0-20 |
| broad-spectrum filter | 0-20 |
| Preservative | 0.5-1 |
| Water | 30-95 |
| (to complete to 100%) | |

In embodiments the sunscreen composition provides for a Sun Protection Factor (SPF) of at least 15, or at least 30, or at least 45, at least 50, or at least 60.

The sunscreen compositions in accordance with the present invention may find numerous applications, at least for protecting the skin against the sun. Methods of use may comprise simply applying the composition(s) onto the skin. Application(s) may be repeated as needed, for instance at different time interval (e.g., every hour, or every 2 h, or every 6 hour, or every 12 hours, or once a day, or twice a day, or thrice a day, or once or twice or thrice a week, etc.).

### Skin Care Compositions

One particular aspect of the invention concerns skin care compositions comprising a biopolymer and/or biopolymer suspension as described herein. In embodiments the biopolymer comprises, or alternatively consists of, a biopolymer composition comprising biopolymer molecules that have been mechanically processed into a stable homogeneous aqueous suspension (e.g., a suspension of insoluble and/or semi-soluble particles stably dispersed within a polar solvent).

Advantageously the skin care compositions may comprise smaller monomeric units of a multimeric biopolymer, such as N-Acetyl glucosamine (GlcNAc) and/or oligomers of NAGs that may exhibit anti-aging properties.

In one embodiment, the skin care composition comprises a biopolymer and at least one of a humectant, an emollient, an occlusive, a ceramide, an emulsifier, a thickening agent, a preservative, an antioxidant, a fragrance, and a functional ingredient. In embodiments, the skin care composition comprises at least a biopolymer, an emulsifier/stabilisers, and a preservative.

In embodiments the humectant is selected from the group consisting of glycerin, urea, hyaluronic acid, salicylic acid, alpha hydroxy acid, propylene glycol, honey, sorbitol, butylene glycol, sodium PCA, allantoin, sodium lactate, dicyanamide, tremella extract, seaweed, aloe vera and mixtures thereof.

In embodiments the emollient is selected from the group consisting of beeswax, butylphthalimide isopropylphthalimide, candelilla, castor, cocoa butter, coconut oil, cyclomethicone, cyclopentasiloxane, dicaprylyl ether, diisopropyl dimer dilinoleate, dimethicone, dipropylene glycol dibenzoate, glycerin, hydrogenated polyisobutene, isocetyl stearoyl stearate, Isododecane, isohexadecane, isoparaffin, isopropyl isostearate, isopropyl palmitate, isopropyl stearate, jojoba oil, lanolin, mineral oil, octyl dodecanol, octyl palmitate, olive oil, palm, pentaerythritol tetraoctanoate, petrolatum, phenyl ethyl benzoate, polyglyceryl-4 oleate, propylene glycol, propylene glycol dibenzoate, propylene glycol isostearate, shea butter, sorbitol, squalane, sunflower, trisiloxane. and mixtures thereof.

In embodiments the occlusive is selected from the group consisting of mineral oil, petrolatum, lanolin, dimethicone, shea butter and mixtures thereof.

In embodiments the ceramide is selected from the group consisting of ceramide EOS, ceramide NS, ceramide NG, ceramide NP, ceramide AP, ceramide EOP, phytosphingosine, sphingosine and mixtures thereof.

In embodiments the emulsifier is selected from the group consisting of sodium lauryl sulfate, sodium dioctyl sulfosuccinate, tragacanthins, lecithin, mustard, soy lecithin, sodium phosphates, diacetyl tartaric acid ester of monoglyceride, sodium stearoyl lactylate, and mixtures thereof.

In embodiments the thickening agent is selected from the group consisting of magnesium aluminium, silicate, silica, bentonite, carbomer, cetyl palmitate, ammonium acryloyldimethyltaurate and mixtures thereof.

In embodiments the preservative is selected from the group consisting of phenoxyethanol, benzyl alcohol, sodium benzoate, potassium sorbate, ethylhexylglycerin, DMDM hydantoin, quaternium-15, imidazolidinyl urea, diazolidinyl urea, polyoxymethylene urea, sodium hydroxymethylglycinate, 2-bromo-2-nitropropane-1,3-diol (bromopol), glyoxal, ethylparaben, butylparaben, methylparaben, propylparaben, isobutylparaben, isopropylparaben, benzylate, benzoic acid, benzyl ester, methylisothiazolinone (MIT; 2-methyl-4-isothiazoline-3-one), methylchloroisothiazolinone (CMIT; 5-chloro-2-methyl-4-isothiazolin-3-one), phenoxyethanol, 2-phenoxyethanol, Euxyl K™ 400 (mixture of phenoxyethanol and 1,2-dibromo-2,4-dicyanobutane), triclosan (TSC), triclocarban (tcc), benzyl alcohol, benzalkonium chloride, citric acid, dehydroacetic acid, essential oils, grapefruit seed extract, lactic acid, levulinic acid, potassium sorbate, sodium dehydroacetate, sodium metabisulfite, sodium salicylate, sorbic acid, vitamin E, zinc pyrithione and mixtures thereof. In embodiments the preservative comprises at least one of Dermosoft™ and Spectrastat™.

In embodiments the antioxidant is selected from the group consisting of vitamin A, vitamin C, vitamin E, vitamin B3, green tea extract, resveratrol, curcumin, lycopene, coenzyme Q10, polyphenols, C. asiatica, tamanu, and mixtures thereof.

In embodiments the fragrance is selected from the group consisting of linalool, citronellol, limonene, geraniol, eugenol, lavender oil (Lavandula angustifolia), rose flower extract Rosa damascene, canina, centifolia, gallica, multiflora), bergamot oil (Citrus bergamia), ylang-ylang oil (Canaga odorata), lemon (Citrus limon), lime (Citrus aurantifolia or Citrus medica), orange (Citrus sinensis), tangerine (Citrus tangerine), peppermint (Mentha piperita), spearmint (Mentha spicata), eucalyptus, cinnamon (Cinnamomum), cinnamal and mixtures thereof.

In embodiments the functional ingredient is selected from the group consisting of biological extracts, vitamins, amino acids, peptides, proteins, essential fatty acids (e.g., alpha hydroxy acids, bets hydroxy acids, beta-glucans and mixtures thereof.

The skin care compositions in accordance with the present invention may find numerous applications, at least for benefiting the skin. For instance, the skin care compositions may provide one or more of the following benefits: moisturizing the skin, improving skin appearance, protecting from environmental damages, fighting the effects of aging, removing wrinkles and/or sunspots.

In embodiments the skin care composition comprises the following ingredients:

| | % (w/w) IN THE |
|---|---|
| FUNCTION(S) | FORMULATION |
| biopolymer | 0.3-10 |
| (dry weight) | |
| humectant | 0-20 |
| emollient | 1-15 |
| occlusive | 0-10 |
| ceramide | 0-5 |
| emulsifier | 0.2-10 |
| thickening agent | 0-5 |
| preservative | 0.5-1 |
| antioxidant | 0-0.5 |
| fragrance | 0-0.2 |
| functional ingredient | 0-10 |
| Water | 30-95 |
| (to complete to 100%) | |

Methods of use may comprise simply applying the composition(s) onto the skin. Application(s) may be repeated as needed, for instance at different time interval (e.g., every hour, or every 2 h, or every 6 hour, or every 12 hours, or once a day, or twice a day, or thrice a day, or once or twice or thrice a week, etc.).

In embodiments, a skin care composition in accordance with the present invention is useful in “smoothing” the skin (e.g., removing wrinkles). In embodiments, the skin care composition provides a decrease of mean Ra parameter (Linear Average Profile Roughness) by an average value of at least 0.1%, or at least 0.25%, or at least 0.5%, or at least 1%, or at least 1.5%, or at least 2%, or at least 2.5%, or at least 3%, or at least 3.5%, or at least 4%, or at least 4.5%, or at least 5%, or between 0.5%-5%, or between 1% to 3%, or about 2.5%, 15 minutes after application on the skin of a human subject. In other embodiments, the skin care composition provides a decrease of mean Ra parameter by the same value mentioned above, but after 10 days of usage (application on the skin once, twice or three times per day, for 10 days).

In embodiments, a skin care composition in accordance with the present invention have waterproof effects, as measured after splashing water 10 times from the product application. In embodiments, a skin care composition has waterproof score greater than or equal to 7, e.g., at least 7, or at least 7.5, or at least 8, or at least 8.5, or at least 9, or at least 9.5.

In embodiments, skin care compositions in accordance with the present invention are dermatologically safe. In embodiments application of the composition on the skin does not cause any undesirable effect such as erythema, desquamation, edema, and vesicles. Preferably, skin care compositions in accordance with the present invention display a Mean Irritation Index (MII) that is very low or nil (e.g., MII of 0 both 15 min and 24 h after application in accordance with a regular patch test (see Example 9 hereinafter). Preferably, skin care compositions in accordance with the present invention may be classified as “not-irritant” and “dermatologically tested for skin compatibility”.

In embodiments, skin care compositions in accordance with the present invention can fully satisfy the requirements of the preservation efficacy test for topically used products, according to USP regulation (e.g., USP 51 and/or USP 61). Preferably the skin care composition can reduce microbial count of various microbial strains, including but not limited to Escherichia coli ATCC 8739, Pseudomonas aeruginosa ATCC 9027, Staphylococcus aureus ATCC 6538, Candida albicans ATCC 10231, and Aspergillus brasiliensis ATCC 16404. Preferably, the skin care composition is effective in reducing microbial count by at least 1 log, or at least 2 log, or at least 3 log, or at least 4 log, or at least 5 log, over 14 days. In embodiments, the skin care composition can be correctly preserved and prevent growth of bacteria for at least 5 days, or at least 10 days, at least 14 days, at least 21 days, at least 28 days or more.

### Hair Care Compositions

One particular aspect of the invention concerns hair care compositions comprising a biopolymer and/or a biopolymer suspension as described herein. In embodiments the biopolymer comprises, or alternatively consists of, a biopolymer composition comprising biopolymer molecules that have been mechanically processed into a stable homogeneous aqueous suspension (e.g., a suspension of insoluble and/or semi-soluble particles stably dispersed within a polar solvent).

In one embodiment, the hair care composition comprises a biopolymer and at least one of a surfactant, a hair conditioning surfactant, a cosurfactant, a cationic conditioning polymer, a conditioning emollient, a thickening agent, an opacifier, a ceramide, a conditioner, a pH buffer, a chelating agent, a preservative, an antioxidant, a fragrance, and a functional ingredient. In embodiments, the skin care composition comprises at least a biopolymer, an emulsifier, a stabiliser, and a preservative.

In embodiments the biopolymer is selected from chitin, chitosan, cellulose and mixtures thereof. In embodiments the biopolymer is a microcrystalline cellulose (e.g., SunSpheres™ BIO SPF Booster), a microfibrillated cellulose (e.g., Exilva™ (FM02-V), Borregaard). It also envisionable to use mixtures of these cellulose.

In embodiments the surfactant is selected from lauryl sulfates (e.g., sodium lauryl sulfate), laureth sulfates (e.g., sodium laureth sulfate), sarcosines (e.g., sodium lauroyl sarcosinate), sulfosuccinates (e.g., sodium dioctyl sulfosuccinate), betaines (e.g., cocamidopropyl betaine), alkylamino acids, poloxamers, amine oxides (e.g., cocamidopropylamine oxide), polyglucosides (e.g., lauryl glucoside) and mixtures thereof.

In embodiments the conditioning surfactant selected from behentrimonium chloride, stearyl dimethylaminopropylamine, cetrimonium chloride, stearalkonium chloride and mixtures thereof.

In embodiments the cationic conditioning polymer is selected from polyquaternium 7, 10, 53, 87, guar hydroxypropyltrimonium chloride, starch hydroxypropyltrimonium chloride, Sensomer™ CI-50 (33% active, Lubrizol), cassia hydroxypropyltrimonium chloride and mixtures thereof.

In embodiments the cosurfactant is selected from cetyl, stearyl alcohol, glyceryl stearate and mixtures thereof.

In embodiments the conditioning emollient is selected from dimethicone, dimethiconol, trisiloxane, amodimethicone, polyricinoleic acid, polyester 37, trimethylolpropane tricaprylate/tricaprate and polyester-11, PPG-3 benzyl ether ethylhexanoate and mixtures thereof.

In embodiments the preservative is selected from caprylyl glycol, hexanediol, ethyl lauroyl arginate HCl, Everguard™ LAE 20 (20% solution in glycerin), benzyl alcohol, glyceryl caprylate, trisodium ethylenediamine disuccinate, sodium benzoate, potassium sorbate, Euxyl™ k 712 preservative (sodium benzoate (and) potassium sorbate (and) aqua-ashland) and mixtures thereof.

In embodiments the thickening agent is selected from sodium chloride, gums, celluloses, polyvinyl alcohol, acrylates copolymer and mixtures thereof.

In embodiments the preservative is selected from isothiazolones (e.g., methylchloroisothiazolinone), cationic surfactant (e.g., benzalkonium chloride) and mixtures thereof. Additional examples of possible preservatives, as well as of possible stabilizers and/or emulsifiers are provided hereinafter.

In embodiments the opacifiers and pearlescent agent is selected from polyglycolesters latexopacifiers, pearlescent color additives and mixtures thereof.

In embodiments the conditioner is selected from quaternary surfactants, (e.g., stearalkonium chloride, cetrimonium chloride, quaterniums, and polyquaterniums (e.g., polyquaternium-10), polyvinylpyrrolidone, humectants, (e.g., glycerin), proteins, silicones (e.g., dimethicone cyclomethicone, dimethicone, and amodimethicone) and mixtures thereof.

In embodiments the pH buffer is selected from citric acid, glycolic acid and mixtures thereof.

In embodiments the chelating agent is EDTA.

In embodiments the active ingredient is selected from anti-dandruff agents (e.g., ketoconazole) zinc pyrithione, sulfur and salicylic acid and mixtures thereof.

In embodiments the additional ingredient is selected from vitamins, fragrances, botanical extracts and mixtures thereof.

The hair care compositions in accordance with the present invention may find numerous applications, for instance in a shampoo, a rinse out cream hair conditioner, a spray on hair conditioner, a body wash, and/or any other products for washing hairs, preventing and/or treating damages to the hair, etc. Methods of use may comprise simply applying the composition(s) into the hair (and/or body) during washing, rinsing with water and drying the hair (and/or). Application(s) may be repeated as needed, for instance at different time interval (e.g., once a day, or twice a day, or once or twice or thrice a week, etc.).

In embodiments the hair care composition is formulated a shampoo (e.g. a conditioning shampoo) and comprises the following ingredients:

| | % (w/w) IN THE |
|---|---|
| FUNCTION(S) | FORMULATION |
| Biopolymer | 0.3-10 |
| (dry weight) | |
| Hair conditioning Surfactant | 1-5 |
| Cationic conditioning polymers | 1-6 |
| Emollient | 0.5-2 |
| Cosurfactant | 0.1-0.4 |
| Pearlescent Agent | 0.5-2 |
| Chelating Agent | 0.1-0.4 |
| pH buffer | 0.1-0.5 |
| Preservative | Up to 1 |
| Water | 30-95 |
| (to complete to 100%) | |

In embodiments the hair care composition is formulated a rinse out cream hair conditioner and comprises the following ingredients:

| | % (w/w) IN THE |
|---|---|
| FUNCTION(S) | FORMULATION |
| Biopolymer | 0.3-10 |
| (dry weight) | |
| Hair conditioning Surfactant | 0.5-2 |
| Cationic conditioning polymers | 1-4 |
| Cosurfactant | 1-7 |
| Humectant | 0-2 |
| Emollients | 0-2 |
| Fragrance | 0.1-0.5 |
| pH buffer | 0-0.5 |
| Preservative | Up to 1 |
| Water | 60-95 |
| (to complete to 100%) | |

In embodiments the hair care composition is formulated a spray on hair conditioner and comprises the following ingredients:

| | % (w/w) IN THE |
|---|---|
| FUNCTION(S) | FORMULATION |
| Biopolymer | 0.3-2 |
| (dry weight) | |
| Hair conditioning Surfactant | 0.1-0.4 |
| Cationic conditioning polymers | 0.1-0.5 |
| Emollient | 0-2 |
| Cosurfactant | 0.1-0.5 |
| pH buffer | 0-0.5 |
| Preservative | Up to 2 |
| Water | 85-95 |
| (to complete to 100%) | |

### Desirable Properties

The cosmetic compositions in accordance with the present invention are preferably stable. Stability at least means that the biopolymer (e.g., fibers, spherical bodies) or any other component of the compositions, does not settle at the bottom. In embodiments the insoluble and/or semi-soluble biopolymer(s) remains in suspension for at least 1 week, or at least 1 month, or at least 6 months, or at least 12 months, or at least 18 months, or at least two years, or at least three years or more.

The cosmetic compositions in accordance with the present invention may be formulated to have a desired viscosity (e.g., viscosity of what is generally referred to as a paste, an ointment, a cream, a lotion, a gel or a milk). In embodiments the viscosity of the compositions/suspensions can be altered by varying the high-shearing conditions and/or mechanical energy to which the biopolymer(s) are submitted. In embodiments the stable homogeneous suspension comprises a viscosity of about 25 mPa to about 85 000 mPa. Table 1 below provides non-limiting examples of desirable viscosity for the compositions/suspensions in accordance with the present invention.

TABLE 1
Examples of desired viscosities

| Desired Consistency | Exemplary viscosity (mPa · s) |
|---|---|
| paste | about 40 000 to about 300 000 |
| ointment | about 20 000 to about 50 000 |
| cream | about 1 500 to about 30 000 |
| lotion | about 800 to about 4 000 |
| gel | about 1 000 to about 40 000 |
| milk | about 20 to about 2000 |

In embodiments, the viscosity the skin care and/or sunscreen compositions in accordance with the present invention may be varied by selecting accordingly the sensory enhancer, film former, emulsifier, emollients, UVB filter, UVA filter, and/or broad-spectrum filter.

The viscosity the cosmetic compositions in accordance with the invention may also be varied in accordance with the ratios or concentration of each of the compound entering into the composition, in addition to the biopolymer. In embodiments the weight ratio of biopolymers:other components (e.g., sensory enhancer, film former, emulsifier, emollients, UVB filter, UVA filter, and/or broad-spectrum filter, etc.) is of about 0.1:20 to about 10:20, or about 0.5:20 to about 3:20, or about 0.75:20, or about 1.0:20, 1.25:20. or about 1.5:20. In embodiments the cosmetic compositions in accordance with the invention comprises about 0.01-10% w/w of biopolymer, or 0.01-5% w/w of biopolymer, or 0.01-2% w/w of biopolymer, or 0.01-1% w/w of biopolymer. In embodiments the cosmetic compositions in accordance with the invention comprises about 0.01% w/w of biopolymer, or about 0.05% w/w of biopolymer, or about 0.1% w/w of biopolymer, or about 0.25% w/w of biopolymer, or about 0.5% w/w of biopolymer, or about 0.75% w/w of biopolymer, or about 1% w/w of biopolymer, or about 1.5% w/w of biopolymer, or about 2.5% w/w of biopolymer, or about 5% w/w of biopolymer.

The cosmetic compositions in accordance with the invention may also be formulated as stable emulsions comprising fatty acids (e.g., C₁₀-C₂₂ fatty acids), an oil and/or a wax, and/or comprising N-Acetyl Glucosamine, and/or emulsifiers and preservatives, and/or additives.

A not limitative list of possible additives includes, but is not limited to, preservatives, stabilizers and emulsifiers (e.g., Cetyl alcohol, Glyceryl stearate, Soy butter, PC90, Tara Gum, PSC3, PEG, Guar, Xanthan gum, Agarose, Sodium Hyaluronate, Tween 80™, Glycerol (humectant), thickeners, dyes, powders (e.g., mica, pigment, chalk), inks, colorants, fragrances, essential oils, extracts (e.g., plant extract(s) such as aloe vera), vitamins (e.g., ascorbic acid), acids (e.g., acetic acid, citric acid, stearic acid), oils (cocoa butter, emu oil, olive oil, shea butter, silicone oil, mineral oil), metal oxides (e.g., zinc oxides), salts (e.g., sea salts, sodium lactate), honey, clay, propenyl glycol, polyethylene glycol, dry ingredient (e.g., rose petal powder, orange peel powder, chamomile flowers, Calendula petals, etc.), allantoin, acetylglucosamine (GlcNAc), waxes (e.g., beeswax), peptides and proteins, pharmaceutical compounds (e.g., N-Acetyl Glucosamine, lidocaine, capsaicin, baclofen, ketamine, methylsulfonylmethane, orphenadrine, tetracaine, amitriptyline, bupivacaine, cyclobenzaprine, doxepin, gabapentin, guaifenesin, acetaminophen, ibuprofen, naproxen, diclofenac, meloxicam, piroxicam, ketoprofen, any NSAIDs), sugars (e.g. glucose, fructose, galactose, etc.), monomers of any of cellulose, starch, chitin, chitosan, alginic acid, collagen, silk, etc. The additive(s) may be added prior, during and/or after the step of high-shearing conditions and/or high mechanical energy.

In embodiments, the additive or stabilizer is selected from the following stabilizers: agar, sodium alginate, carrageenan, guar, konjac, tragacanth, locust bean gum, psyllium, tara gum, fenugreek gum, xanthan gum, abietic acid, acetyl mannosylerythritol lipid, acrylamide/sodium acryloyldimethyltaurate copolymer, acrylates/aminoacrylates/C10-30 alkyl peg-20 itaconate copolymer, acrylates/C10-30 alkyl acrylate crosspolymer, acrylates/C5-8 alkyl acrylate copolymer, acrylates/stearyl methacrylate copolymer, acrylates/vinyl isodecanoate crosspolymer, acrylates/vinyl neodecanoate crosspolymer, acrylic acid/stearyl acrylate copolymer, acrylic acid/stearyl methacrylate/dimethicone methacrylate copolymer, bis-acryloyl poloxamer, alcaligenes polysaccharides, alcohols C9-11, allyl methacrylates crosspolymer, sweet almond oil polyglyceryl-4 esters, aluminum behenate, aluminum caprylate, aluminum dicetyl phosphate, aluminum dilinoleate, aluminum dimyristate, aluminum distearate, aluminum isostearate, aluminum isostearates/laurates/palmitates, aluminum isostearates/laurates/stearates, aluminum isostearates/myristates, aluminum isostearates/palmitates, aluminum isostearates/stearates, aluminum lanolate, aluminum monostearate, aluminum myristate, aluminum myristates/palmitates, aluminum/magnesium hydroxide stearate, ammonium acryloyldimethyltaurate/steareth-25 methacrylate crosspolymer, ammonium acryloyldimethyltaurate/steareth-8 methacrylate copolymer, ammonium acryloyldimethyltaurate/vinyl formamide copolymer, ammonium alginate, ammonium phosphatidyl rapeseedate, ammonium polyacryloyldimethyl taurate, ammonium shellacate, amodimethicone glycerocarbamate, AMP-C8-18 perfluoroalkylethyl phosphate, aphanothece sacrum polysaccharide, arachidyl alcohol, astragalus gummifer gum, astragalus gummifer root extract, avocadamide DEA, babassu acid, crosslinked bacillus/glucose/sodium glutamate ferment, dextro,laevo-batyl alcohol, hydrolyzed beeswax, synthetic beeswax, behenyl alcohol, bentonite, benzalkonium montmorillonite, benzalkonium sepiolite, bittern emulsion stabilising, brassica alcohol emollients, brassicyl isoleucinate esylate, butendiol/vinyl alcohol copolymer, butoxyhydroxypropyl cetyl hydroxyethylcellulose, butter decyl esters, butyl acrylate/isopropylacrylamide/peg-18 dimethacrylate crosspolymer, butyl babassuate, butylene glycol cocoate, butylene glycol isostearates, C1-5 alkyl galactomannan, C12-13 alcohols, C12-14 sec-pareth-3, C12-14 sec-pareth-5, C12-14 sec-pareth-7, C12-14 sec-pareth-8, C12-14 sec-pareth-9, C12-14 sec-pareth-12, C12-14 sec-pareth-15, C12-14 sec-pareth-20, C12-14 sec-pareth-30, C12-14 sec-pareth-40, C12-14 sec-pareth-50, C12-15 alcohols, C12-16 alkyl peg-2 hydroxypropyl hydroxyethyl ethylcellulose, C12-18 alkyl glucoside, Hydrogenated C12-18 triglycerides, C14-15 alcohols, C14-18 glycol, C14-22 alcohols, C15-18 glycol, bis-C16-18 alkyl glyceryl undecyl dimethicone, C18-22 alkyl peg-25 methacrylate/diethylaminoethyl methacrylate copolymer, C18-30 glycol, C18-38 alkyl hydroxystearoyl stearate, C20-22 alcohols, C20-30 glycol, C20-40 alcohols, C20-40 alkyl crylene, C22-24 pareth-33, bis-C24-28 hydroxyalkyl olivoyl glutamate, C28-52 olefin/undecylenic acid copolymer, C30-50 alcohols, calcium carboxymethyl cellulose, calcium carrageenan, calcium laurate, calcium myristate, calcium polyglutamate crosspolymer, calcium potassium carbomer, calcium saccharate, calcium starch octenylsuccinate, calcium stearate, Callitris quadrivalvis resin, candelilla cera, candelilla wax, candelilla/jojoba/rice bran polyglyceryl-3 esters, Cannabis sativa seed oil glycereth-8 esters, caprylyl dimethicone ethoxy glucoside, caprylyl/capryl wheat bran/straw glycosides, carbomer 934, carboxymethyl cellulose acetate butyrate, carboxymethyl hydroxyethyl cellulose, carboxymethyl hydroxypropyl guar, carnauba wax, Carthamus tinctorius oleosomes, hydrogenated castor oil behenyl esters, castor oil phosphate, hydrogenated castor oil stearyl esters, hydrogenated castor oil/sebacic acid copolymer caprate/caprylate, cellulose acetate propionate carboxylate, hydrolyzed cellulose gum, cellulose microcrystalline, ceramide NS/peg-8/succinic acid copolymer, Ceratonia siliqua gum, ceresin, ceteareth-6 olivate, cetostearyl alcohol, cetyl alcohol, cetyl dimethicone peg-7 acetate, cetyl dodecenylsuccinate, cetyl hydroxyethyl cellulose, cetyl peg/ppg-7/3 dimethicone, bis-cetyl/peg-8 cetyl peg-8 dimethicone, chitosan lauramide succinimide, chitosan lauroyl glycinate, cholesterol/hdi/pullulan copolymer, citrus aurantium dulcis peel extract, citrus aurantium sinensis fiber, cocamide, cocamide DEA, cocamide MEA, cocamide MIPA, cocamidopropyl lauryl ether, cocoa butter glyceryl esters, coconut alcohol, coconut oil methylpropanediol esters, hydrolyzed corn starch hydroxyethyl ether, Cyamopsis tetragonoloba gum, decyl castorate, decyl glucoside, decyl hempseedate, 7-dehydrocholesterol, dehydroxanthan gum, dicapryl sodium sulfosuccinate, diethylene glycol/hydrogenated dimer dilinoleic acid copolymer, digalactosyl glyceryl linoleate/palmitate/oleate, diglycerin/dilinoleic acid/hydroxystearic acid copolymer, dihydrolanosterol, dihydroxyethyl cocamine oxide, dihydroxyethyl lauramine oxide, diisotridecyl lauroyl glutamate, dilauryl maleate/C20 olefin copolymer, dimaltosyl cyclodextrin, hydrogenated dimer dilinoleyl/dimethylcarbonate copolymer, dimethicone crosspolymer, dimethicone ethoxy glucoside, dimethicone/lauryl dimethicone/bis-vinyldimethicone crosspolymer, dimethicone/peg-15 crosspolymer, dimethyl capramide, dimethyl cocamine, dimethyl lauramine isostearate, dioleyl phosphate, dipropylene glycol isobornyl ether, dodecylhexadecanol, bis-ethoxydiglycol cyclohexane 1,4-dicarboxylate, ethyl hydroxyethyl cellulose, bis-ethyl ppg-behenate dimonium methosulfate, bis-(ethyl ppg-3 behenate) dimonium methosulfate, ethylene vinyl acetate copolymer, ethylene/acrylic acid copolymer, ethylene/sodium acrylate copolymer, feruloyl soy glycerides, perfluorocyclohexylmethanol, perfluoroheptane, perfluoromethylcyclohexane, perfluoromethyldecalin, ghatti gum, glucose pentaacetate, alpha-dextro-glucose pentaacetate, glycereth-7 malate, glycereth-8 hydroxystearate, glycereth-7 benzoate, hydrogenated glyceryl abietate, glycol cetearate, acetylated glycol stearate, glycosyl trehalose, grape seed oil glycereth-8 esters, grape seed oil polyglycerin-6 esters, maleated hexene/propylene copolymer, hydroxquinoline sulfate, hydroxyapatite, hydroxybutyl methyl cellulose, bis-hydroxyethoxypropyl dimethicone beeswax esters, bis-hydroxyethoxypropyl dimethicone isostearate, hydroxyethyl acrylate/sodium acryloyldimethyl taurate copolymer, hydroxyethyl cellulose, hydroxyethyl isostearyloxy isopropanolamine, hydroxypropyl cellulose, hydroxypropyl guar gum, hydroxypropyl methyl cellulose, hydroxypropyl xanthan gum, hydroxypropyltrimonium inulin, hydroxypropyltrimonium xanthan gum, hydroxystearic/linolenic/linoleic polyglycerides, hydroxystearic/linolenic/oleic polyglycerides, inulin lauryl carbamate, synthetic japan wax, jojoba oil glycereth-8 esters, hydrogenated lanolin alcohol, lanolinamide DEA, lauryl alcohol, lauryl alcohol diphosphonic acid, lauryl dodecenylsuccinate, lauryl/myristyl wheat bran/straw glycosides, hydrogenated lime seed oil, magnesium alginate, maltitol laurate, maltodextrin, methoxy peg-22/dodecyl glycol copolymer, methoxy peg/ppg-25/4 dimethicone, methyl cellulose, methyl vinyl ether-maleic anhydride copolymer, montmorillonite, myrist/palmitamidobutyl guanidine acetate, myristyl alaninate, myristyl alcohol, oleic/linoleic/linolenic polyglycerides, olive alcohol, hydrogenated olive oil caprylyl esters, hydrogenated olive oil cetyl esters, hydrogenated olive oil decyl esters, hydrogenated olive oil hexyl esters, hydrogenated olive oil lauryl esters, hydrogenated olive oil myristyl esters, hydrogenated olive oil stearyl esters, hydrogenated orange seed oil, ozokerite, palm kernel amide DEA, palm kernel amide MEA, palm kernelamide MIPA, palmamide DEA, palmamide MEA, palmamide MIPA, peanutamide MEA, peanutamide MIPA, pectin, tris(peg-2 phenylalanylcarboxamido) cyclohexane, peg-2 tallowamide DEA, peg-4 peg-12 dimethicone, peg-5 pentaerythrityl dimethylol propionate-2 dendrimer, peg-5 pentaerythrityl dimethylol propionate-3 dendrimer, peg-5 pentaerythrityl dimethylol propionate-4 dendrimer, peg-7 propylheptyl ether, peg-7m, peg-8 dimethicone/polysorbate 20 crosspolymer, peg-8 propylheptyl ether, peg-9m, peg-12 carnauba, peg-12 glyceryl linoleate, peg-14m, peg-20m, peg-23m, peg-65m, peg-90m, peg-100/IPDI copolymer, peg-114 polylactic acid, peg-115m, peg-160m, peg-180m, peg-400, peg-45/dodecyl glycol copolymer, peg-450, peg-500, peg/ppg-10/3 oleyl ether dimethicone, peg/ppg-100/70 tocopheryl ether, bis-peg/ppg-15/5 dimethicone, peg/ppg-18/18 isostearate, peg/ppg-18/18 laurate, peg/ppg-2/5 tocopheryl ether, peg/ppg-20/23 dimethicone, bis-peg/ppg-20/5 peg/ppg-20/5 dimethicone, peg/ppg-2000/200 copolymer, peg/ppg-23/6 dimethicone, peg/ppg-30/10 tocopheryl ether, peg/ppg-5/10 tocopheryl ether, peg/ppg-5/20 tocopheryl ether, peg/ppg-5/30 tocopheryl ether, peg/ppg-50/20 tocopheryl ether, peg/ppg-6/4 dimethicone, peg/ppg-70/30 tocopheryl ether, peg/ppg-8/3 laurate, pentadecyl alcohol, petrolatum wax microcrystalline, phosphatidic acid, phosphatidyl serine, phosphatidylglycerol, pineamidopropyl betaine, poly C10-30 alkyl acrylate, polyacrylate crosspolymer-4, polyacrylate crosspolymer-6, polyacrylate crosspolymer-11, polyacrylate crosspolymer-14, polyacrylate-10, polyacrylate-11, polyacrylate-27, polyacrylate-28, polyacrylic acid, polyester-14, polyester-15, polyethylene/isopropyl maleate/MA copolyol, polyglyceryl-2 diisostearate/ipdi copolymer, polyglyceryl-3 sunflowerseedate/citrate crosspolymer, polyglyceryl-4 diisostearate/polyhydroxystearate/sebacate, polyglyceryl-6 behenate, polypropanediol, polypropylene terephthalate, polyquaternium crosspolymer-2, polyquaternium-65, polyquaternium-83, polyquaternium-102, polyquaternium-103, polysilicone-25, polyurethane-29, polyvinyl acetate, polyvinyl pyrrolidone, potassium alginate, potassium behenoyl hydrolyzed rice protein, potassium behenoyl hydroxyproline, potassium carbomer, potassium carrageenan, potassium stearoyl hydrolyzed rice protein, potassium undecylenoyl alginate, potassium undecylenoyl carrageenan, potassium undecylenoyl hydrolyzed corn protein, potassium undecylenoyl hydrolyzed soy protein, potassium undecylenoyl hydrolyzed wheat protein, hydrolyzed potato tuber extract, ppg-4 jojoba alcohol, ppg-4 laureth-2, ppg-4 laureth-5, ppg-6-laureth-3, ppg-20 tocophereth-5, ppg-10 jojoba acid, ppg-2-buteth-2, propyl ester of PVM/MA copolymer, Prunus amygdalus dulcis oil unsaponifiables, Pseudozyma epicola/Camellia sinensis seed oil/glucose/Glycine soja meal/malt extract/yeast extract ferment filtrate, PVP montmorillonite, PVP/decene copolymer, Pyrus malus fiber, quaternium-90 sepiolite, rhamnolipids, sclerotium gum, hydrogenated sesame seed oil, sesquiethoxytriethanolamine, sesquioctyldodecyl lauroyl glutamate, shea butter glycerides, silica dimethyl silylate, silica silylate, beta-sitosterol, sodium acrylate/acryloyldimethyltaurate/dimethylacrylamide crosspolymer, sodium acrylate/sodium acryloyldimethyl taurate copolymer, sodium acrylate/sodium acryloyldimethyl taurate/acrylamide copolymer, sodium acrylate/vinyl alcohol copolymer, sodium acrylates/vinyl isodecanoate crosspolymer, sodium acryloyldimethyl taurate/acrylamide/VP copolymer, sodium acryloyldimethyltaurate/VP crosspolymer, sodium arachidate, sodium C4-12 olefin/maleic acid copolymer, sodium carbomer, sodium carboxymethyl cellulose, sodium carboxymethyl dextran, sodium carboxymethyl starch, sodium carrageenan, sodium cellulose sulfate binding, sodium cocoyl barley amino acids, sodium cocoyl/stearoyl (alanine/arginine/asparagine/aspartic acid/glutamic acid/glutamine/glycine/histidine, sodium cyclodextrin sulfate, sodium dextrin octenylsuccinate, sodium laneth sulfate, sodium polyacrylate, sodium polyacrylate starch, sodium polyacryloyldimethyl taurate, sodium polygamma-glutamate, sodium polygamma-glutamate crosspolymer, sodium polyglutamate crosspolymer, sodium polymethacrylate, sodium polynaphthalenesulfonate, sodium polystyrene sulfonate, sodium starch octenyl succinate, sodium styrene/MA copolymer, sodium tocopheryl phosphate antioxidants, sodium trehalose octenylsuccinate, sodium/TEA-undecylenoyl alginate, sodium/TEA-undecylenoyl carrageenan, sorbitan palmate, soy protein phthalate, soyamide DEA, Sparassis crispa extract, starch hydroxypropyltrimonium chloride, iso-steareth-200 palmitate, stearic acid, stearyl alcohol, stearyl glycol, stearyl vinyl ether/MA copolymer, Sterculia urens gum, stigmasteryl chloride, stigmasteryl nonanoate, stigmasteryl succinate, styrene/ma copolymer, sucrose polypalmate, sunflower seed oil ethyl ferulate esters, sunflower seed oil polyglyceryl-10 esters, sunflower seed oil polyglyceryl-6 esters, tallow alcohol, tallow amide cosmetic agents, Tamarindus indica seed gum, TEA-alginate, TEA-dextrin octenylsuccinate, tetradecyleicosanoic acid, tetradecyloctadecanoic acid, tetradecyloctadecyl behenate, tetradecyloctadecyl myristate, tetradecyloctadecyl stearate, tetrasodium etidronate, Theobroma grandiflorum seed butter glyceryl esters, tocopheryl succinate methylglucamide, Tremella fuciformis polysaccharide, triacontene/VP copolymer, 1-Tridecanol, tripropylene glycol, undeceth-40, undecylenoyl inulin, undecylenoyl xanthan gum, vinyl alcohol/vinylformamide copolymer, bis-vinyl dimethicone/dimethicone copolymer, bis-vinyldimethicone/peg-10 dimethicone crosspolymer, hydrogenated microcrystalline wax hydrotreated, welan gum, xanthan gum, zinc undecylenoyl hydrolyzed wheat protein.

The cosmetic compositions in accordance with the invention may also comprise microbial ingredients (e.g., filtrates, extracts and/or ferment from Saccharomyces, Lactobacillus) and/or microbial-based formulation(s) such as Lallemand's Lallhair® P&R, Lallbiome™ 300, Lallbiome™ 201, Lallskin® Hydra, and/or Lallskin® Zn.

The cosmetic compositions in accordance with the invention may also comprise crosslinked hyaluronic acid. This compound could be used, for instance, as a thickener to make a clear moisturizing hydrogel. Particular examples include, but art not limited to Hyacross™ Hyaluronic Acid Microbeads TG300 (sodium hyaluronate crosspolymer, Bloomage Biotechnology Corp. Ltd.) and Hylasome™ EG10 (sodium hyaluronate crosspolymer, Vantage Personal Care).

### Methods of Manufacture and Substitution of Microplastics

Another aspect of the present invention concerns methods of manufacturing cosmetic compositions (e.g., sunscreen, skin care and/or hair care compositions) as defined herein. A related aspect concerns the use of a biopolymer as described herein for replacing microplastics in cosmetic compositions.

In one embodiment, the method comprises substituting at least a portion of microplastics that may be present in cosmetic compositions with a biopolymer, e.g., a biopolymer suspension, as defined herein. In embodiments the biopolymer suspension comprises a suspension of insoluble and/or semi-soluble particles stably dispersed within a polar solvent.

In one embodiment, the method of manufacturing the sunscreen composition, and/or method for replacing microplastics in a sunscreen composition, comprises: (1) providing a biopolymer suspension as defined herein (e.g., a suspension of nano-size insoluble and/or semi-soluble particles stably dispersed within a polar solvent); and (2) mixing the biopolymer suspension with at least one of a sensory enhancer, a film former, an emulsifier, an emollient, a UVB filter, a UVA filter, and a broad-spectrum filter.

In one embodiment, the method of manufacturing the skin care composition, and/or method for replacing microplastics in a skin care composition, comprises: (1) providing a biopolymer suspension comprising a suspension of insoluble and/or semi-soluble particles stably dispersed within a polar solvent; (2) mixing the biopolymer suspension with at least one of an humectant, an emollient, an occlusive, a ceramide, an emulsifier, a thickening agent, a preservative, an antioxidant, a fragrance, and a functional ingredient.

In one embodiment, the method of manufacturing the hair care composition, and/or method for replacing microplastics in a hair care composition, comprises: (1) providing a biopolymer suspension comprising a suspension of insoluble and/or semi-soluble particles stably dispersed within a polar solvent; (2) mixing the biopolymer suspension with at least one of a surfactant, a thickening agent, an opacifier, a ceramide, a conditioner, a pH buffer, a chelating agent, a preservative, an emulsifier, a stabiliser, an antioxidant, a fragrance, and a functional ingredient.

In embodiments the method of manufacturing the cosmetic composition, and/or method for replacing microplastics in a cosmetic composition, comprises adding ingredients in sequence. The method of manufacturing may also comprise combining together water-based ingredients, combining together oil-based ingredients and the mixing both phases together. Water could be added to oil or oil added to water. The emulsifier, if any could be in either phase. Additional water or oil could also be added during the mixing process(es). Of course, multiple variations are possible, depending of the cosmetic product to be manufactured, the desired characteristics of the final cosmetic product, etc. It is within the skills of the person in the art to test and identify suitable manufacturing process and steps.

Those skilled in the art will recognize, or be able to ascertain, using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further or specifically limiting.

### EXAMPLES

### Example 1: Mineral Filter Sunscreen

Table 2 and Table 3 provide non-limiting examples of envisioned sunscreen compositions.

TABLE 2
Mineral Filter Sunscreen

| Ingredient # | Trade Name | Quantity (% w/w) |
|---|---|---|
| 1 | Water | 31.90 |
| 2 | Xanthan Gum | 0.50 |
| 3 | Glycerin USP | 2.50 |
| 4 | Biopure ™ GLDA | 0.10 |
| 5 | Dermofeel ™ NC | 5.00 |
| 6 | Eumulgin ™ SG | 1.00 |
| 7 | Polyaquol ™ VO4 | 0.10 |
| 8 | UV-CUT ZNO ™ 65 CC | 38.00 |
| 9 | Kester ™ K60 P | 1.00 |
| 10 | Lexfilm ™ SUN | 5.00 |
| 11 | Dub Zenoat ™ | 3.00 |
| 12 | Vitamine E | 0.50 |
| 13 | Geogard ™ ECT | 1.00 |
| 14 | Sepimat P ™ | 2.00 |
| 15 | Biopolymer (chitosan) | 5.00 |
| 16 | Citric Acid in solution (50% | 0.40 |
| | w/w) | |
| 17 | Water | 2.00 |

TABLE 3
Chemical Filter Sunscreen

| Ingredient # | Trade Name | Quantity (% w/w) |
|---|---|---|
| 1 | Water | 48.90 |
| 2 | Glycerin USP | 2.50 |
| 3 | Xanthan Gum | 0.50 |
| 4 | Biopure ™ GLDA | 0.10 |
| 5 | Eusolex ™ OCR Octocrylene | 3.00 |
| 6 | Parsol ™ EHS Octisalate | 5.00 |
| 7 | Finsolv ™ TPP | 2.00 |
| 8 | Lexfilm ™ SUN | 5.00 |
| 9 | Eusolex ™ 9020 Avobenzone | 3.00 |
| 10 | Eusolex ™ HMS Homosalate | 10.00 |
| 11 | DUB Zenoat ™ | 2.50 |
| 12 | Dermofeel ™ NC | 5.00 |
| 13 | Eumulgin ™ SG | 1.00 |
| 14 | Vitamine E | 0.50 |
| 15 | Geogard ™ ECT | 1.00 |
| 16 | Sepimat P ™ | 2.00 |
| 17 | Biopolymer (chitosan) | 5.00 |
| 18 | Water | 3.00 |

In these compositions, the biopolymer composition is a chitosan suspension. Such composition may be prepared as follows. The chitosan suspension is produced with a 1.5 L Supermill Plus™ with 1.25 mm zirconia beads in a 20 L batch by milling with the general milling conditions of 2400 FPM (feet per minute) rotation speed with a pump flow rate of 8 GPH (gallons per hour) using 956 mL of 1.25 mm zirconia beads, where 20 liters of slurry were processed in a 10% solids content (2.2:20) with chitosan powder with particles had a D99 19 μm, D50 of 5 μm and D10 of 2 μm.

An envisioned manufacturing process for these sunscreen compositions is as follows.

### PHASE A

STEP 1—In the main tank, put ingredient #1.
STEP 2—In a stainless container put ingredients #2 and #3, stir until the mixture is homogeneous. Add the mixture to the main tank and stir until it is homogeneous.
STEP 3—While stirring, add ingredient #4 in the main tank. Stir until the mixture is homogeneous. Heat to 75° C.±2° C. Once the temperature is reached, stop heating and maintain the temperature. Stir until the mixture is homogeneous.

### PHASE B

STEP 4—In a stainless container put the ingredients #5, #6, #7, #8, #9, #10, #11 and #12. Heat to 75° C.±2° C. Once the temperature is reached, stop heating and maintain the temperature. Stir until the mixture is homogeneous.

### PHASE A+B

STEP 5—While stirring add the mixture from step 4 to the main tank. Stir until the mixture is homogeneous. Start cooling and keep stirring until temperature reaches 40° C.±2° C.

### PHASE A+B+C

STEP 6—When the temperature in the main tank is below 40° C.±2° C., add one by one, ingredients #13, #14, #15, #16 and #17. Stir between each addition and until the mixture is homogeneous. Keep stirring until the temperature is 25° C.±2° C.

### Example 2: SPF 30 Sunscreen Product

Table 4 provides a non-limiting example of an envisioned sunscreen compositions providing a SPF 30 protection.

TABLE 4
SPF 30 product

| Ingredient(s) | Trade Name | Quantity (% w/w) |
|---|---|---|
| A | Deionized water | 69.9 |
| | Sodium Phytate | 0.1 |
| | Glycerin | 3.0 |
| B | Chitosan | 2.0 |
| | Stearic acid | 2.0 |
| C | Avobenzone | 3.3 |
| | Octocrylene | 7.0 |
| | Octyl Salicylate | 5.0 |
| | C12-15 Alcohol benzoate | 5.0 |
| | Dracorin GOC | 2.0 |
| D | Caprylyl Glycol | 0.5 |
| | Hexanediol | 0.5 |

An envisioned manufacturing process for this sunscreen composition is as follows. Wet ball mill B into A until thick and uniform. Mill C to AB using a roto/stator mill. Switch to sweep mixing and add D to ABC.

### Example 3: Zinc Oxide Formulation (SPF 30+) Water in Oil Emulsion

Table 5 provides a non-limiting example of an envisioned sunscreen compositions comprising zinc oxide and providing a SPF 30+ protection.

TABLE 5
Zinc oxide SPF 30+ product

| Ingredient(s) | Trade Name | Quantity (% w/w) |
|---|---|---|
| A | Deionized water | 50.8 |
| | SunSpheres Bio ™ Booster | 0.5 |
| B | Zinc Sulfate | 0.5 |
| | Zemea ™ (Propanediol) | 2.0 |
| C | Dermol ININ ™ | 12.0 |
| | Solastay ™ S-1 | 3.0 |
| | Castor oil | 1.2 |
| | Citrol ™ DPHS | 3.0 |
| | HallBrite ™ BHB | 7.0 |
| | Dermol ™ TDSA | 5.0 |
| | Tegocare ™ | 2.0 |
| D | Zano 10 Plus ™ | 12.0 |
| E | Caprylyl Glycol | 0.5 |
| | Hexanediol | 0.5 |

An envisioned manufacturing process for this sunscreen composition is as follows. Wet ball mill A and add B. Heat AB to 70° C. Mill D into C using a roto stator mill at 10K rpms for 5 minutes, heat CD to 70° C. Mill CD into ABC using a roto stator mill for 5 minutes at 5K rpms. Cool batch using side sweep mixing and add F at 45° C. Cool to 30° C.

### Example 4: Rinse Out Cream Hair Conditioner

Table 6 provides a non-limiting example of an envisioned rinse out cream hair conditioner.

TABLE 6
Rinse out cream hair conditioner

| Ingredient(s) | Trade Name | Quantity (% w/w) |
|---|---|---|
| A | Exilva ™ (FM02-V) | 5.0 |
| | Deionized water | 88.5 |
| B | Behentrimonium Chloride | 1.0 |
| | Cetyl alcohol | 1.0 |
| | Stearyl alcohol | 1.0 |
| | Polyricinoleic acid | 2.0 |
| C | Everguard ™ LAE 20 | 2.0 |
| | Caprylyl Glycol | 0.5 |

An envisioned manufacturing process for this sunscreen composition is as follows. Wet ball mill A. Heat A and B to 80° C. Homogenize B into A using a roto stator mill for 5 minutes at 5K rpms. Propellor mix AB and cool. Add C at 45° C. Cool to 30° C.

### Example 5: Spray on Hair Conditioner

Table 7 provides a non-limiting example of an envisioned rinse out cream hair conditioner.

TABLE 7
Spray on hair conditioner

| Ingredient(s) | Trade Name | Quantity (% w/w) |
|---|---|---|
| A | Chitosan | 1.0 |
| | Deionized water | 94.5 |
| B | Merquat ™ 550 PR | 2.0 |
| | (9.3% solution polyquaternium 7, | |
| | Lubrizol) | |
| C | Everguard ™ LAE 20 | 2.0 |
| | Caprylyl Glycol | 0.5 |

An envisioned manufacturing process for this sunscreen composition is as follows. Wet ball mill A. Propellor mix B into A. Propellor mix C into AB.

### Example 6: Conditioning Shampoo

Table 8 provides a non-limiting example of an envisioned conditioning shampoo.

TABLE 8
Conditioning Shampoo

| Ingredient(s) | Trade Name | Quantity (% w/w) |
|---|---|---|
| A | Exilva ™ (FM02-V) | 5.0 |
| | Deionized water | 63.7 |
| | Trisodium thylenediamine Disuccinate | 0.1 |
| B | Sodium Laureth 2 Sulfate (70%) | 8.6 |
| | Cocoamidopropyl Betaine (30%) | 20.0 |
| C | TEGO Pearl ™ N 300 (Evonik) | 1.0 |
| D | Sensomer ™ CI-50 | 1.0 |
| E | Euxyl ™ k 712 preservative | 1.0 |

An envisioned manufacturing process for this sunscreen composition is as follows. Wet ball mill A. Sweep mix B into A. Add C. Add D. Add E.

### Example 7: Reduction of Wrinkles and Fine Lines—Cellulose/Chitosan-Based Composition

In this study, the objective was to evaluate reduction of wrinkles and fine lines reduction using a cellulose/chitosan-based composition in accordance with the invention comprising 5% w/w mushroom chitosan, 0.4% w/w cellulose, 5% w/w Dermosoft™ and water [Batch DEF23041801]. Briefly the chitosan and cellulose were wet mill with water for 30 minutes (agitator pressure 7-9 PSI; agitator speed 3000 rpm). The Dermosoft™ preservative was added at 30 minutes, and the milling was continued for another 60 minutes. The temperature of the mixture was maintained between about 30° C.-40° C.

The above cellulose/chitosan-based composition was evaluated using standard guidelines by an independent third-party laboratory. Briefly, 1.5 g of the composition was applied twice a day (morning and evening) on the volar surface of the face of 12 volunteers for 10 days. Measurements and pictures were taken before applying the product (T0), 15 minutes after the first application (T15 Min) and after 10 days of product usage at (T10Days±2days). 3D images were used to monitor the effects of the product with the VisioTOP-300®+AEVA-V4 software. Skin point analyzed at different times were made on a randomized side of the face for each time of analysis. The area was evaluated in each macrophotograph using the VisioTOP-300®. The most representative parameters for the anti-wrinkle efficacy evaluation, i.e., Ra and Rz were also measured by the instrument, where Ra is the linear average profile roughness and Rz is the average maximum profile height difference.

The results were very positive in improving crow's feet roughness and wrinkles. Indeed, the study observed a statistically significant reduction of winkles upon application of the tested cellulose/chitosan-based composition. The mean decrease was 4.1% was after only 15 min and 5.7% after 10 days.

Furthermore, the cellulose/chitosan-based composition was also useful in “smoothing” the skin. The mean Ra parameter (Linear Average Profile Roughness) demonstrated a decrease by an average value equal to −2.2% after 15 minutes of product application. After 10 days of usage, with an average value equal to −3.0%. For both timepoint, the decrease was considered statistically significant with respect to the mean value measured before application.

Accordingly, these results clearly demonstrate that biopolymer compositions in accordance with the present invention are active and useful in reducing both wrinkles and fine lines.

### Example 8: Cellulose Waterproof Testing

The waterproof effect of a cellulose biopolymer composition in accordance with the present invention was evaluated by an independent third-party laboratory. The composition was prepared by ball milling, and it comprised 5% w/w cellulose and water [Batch: CMC B2AC, 450 G]. Briefly, cellulose was pre-milled in isopropanol alcohol using a 1.5 L Supermill PLUS™ horizontal mill for 23 hours (milling speed of 2750 FPM, 1.25 mm zirconia beads). The resulting product was then wet milled using a 1.5 L Supermill PLUS™ horizontal mill at a flow rate of 11 Gallons per hour at a mill speed of 2400 FPM, PSI 3 using 1.25 mm zirconia beads.

Briefly, the composition was applied on the forearm area of 14 volunteers and the waterproof effect of the composition was evaluated by specialized technicians after splashing water. During the evaluation the technicians gave a score from 1 to 10 on the persistence of the composition after splashing water 10 times from initial application.

Typically, a tested product is considered waterproof after splashing water 10 times from the product application if the mean of these scores is greater than or equal to 7. In the present case, a mean score equal to 7.6 out of 10 was given to the composition persistence. The present objective evaluation, which was conducted by specialized personnel at a third-party laboratory, thus demonstrated that cosmetic biopolymer compositions according to the present invention have waterproof effects.

### Example 9: Cellulose Patch Test

A test was carried out by an independent an independent third-party laboratory to assess local skin tolerance of a cellulose biopolymer composition in accordance with the present invention. The composition was of the same batch than Example 8 (i.e., prepared by ball milling, and it comprised 5% w/w cellulose and water [Batch: CMC B2AC, 450 G].

Briefly, about 0.07-0.1 ml of test product (i.e., cellulose biopolymer composition) was applied on the intact skin of the back of adult volunteers. The product was kept in contact with the skin for 48 hours under semi-occlusive patch. Along with the test product a “negative” control empty patch was also applied. Effects caused by the application of the test product were observed 15 minutes and 24 hours after removal of the patch to assess skin reactions, considering parameters such as erythema, desquamation, edema, and vesicles, with severity scores ranging from 0 to 3.

The Mean Irritation Index (MII) of the test product was 0 at both time periods (15 min and 24 h after patch removal). As such, the test product was classified as “not-irritant” and “dermatologically tested for skin compatibility”, according to the reference standard (data not shown). These results thus confirm that cosmetic biopolymer compositions according to the present invention are dermatologically safe.

### Example 10: Skin Barrier Effect and Water Resistance Evaluation—Cellulose/Chitosan-Based Composition

In this study, the objective was to demonstrate the waterproof effect of a cellulose/chitosan-based composition in accordance with the invention. The cellulose/chitosan-based composition was prepared by wet milling in accordance with the details provided at Example 7 (i.e., 5% w/w mushroom chitosan, 0.4% w/w cellulose, 5% w/w Dermosoft™ and water) [Batch DEF23041801].

The evaluation involved thirteen (13) volunteers and two specialized technicians. To assess the waterproof effect, a baseline pigment called Timica® Terra Brown was used for comparison with the cellulose-based composition under examination. The evaluation process included splashing water on the forearm area of the volunteers after applying the tested product (i.e., cellulose/chitosan-based composition as defined above). During the evaluation, two technicians assigned scores ranging from 1 to 10 based on the persistence of the tested product after splashing water 10 times. If the mean of these scores is equal to or greater than 7, the product is considered waterproof after splashing water 10 times from the initial application.

The two specialized technicians gave the tested product a mean score of 9.1 out of 10, for persistence. This high score indicates a high level of waterproof effect. Accordingly, biopolymer compositions in accordance with the present invention can withstand water splashing after 10 applications.

The study further concluded that the tested product provided “a significant decrease in the Transepidermal water loss (TEWL) values on the skin where the product was applied.” These results further confirm that biopolymer compositions in accordance with the present invention have an important skin barrier effect.

### Example 11: Cellulose Challenge Test

The “Challenge Test” is a valuable method used to assess efficacy of preservative systems in non-sterile cosmetics, drugs, and similar products. The test involves artificially contaminating a product in the laboratory to mimic the microbial pollution the product may encounter during manufacturing, storage, and consumer use. This test provides crucial information about a product's ability to resist microbial attacks and maintain stability. As cosmetics and similar products are not required to be sterile, they can have a certain level of environmental microbial contamination that needs to be controlled through proper preservation. The Challenge Test goes beyond typical environmental contamination by introducing high concentrations of microorganisms, allowing for the evaluation of their growth reduction at different time points. The test follows the guidelines outlined in the U.S. Pharmacopoeia and is preceded by an examination of the product's total microbial load.

The inoculum for the present Challenge Test consisted of various microbial strains at different concentrations. The strains used included Escherichia coli ATCC 8739, Pseudomonas aeruginosa ATCC 9027, Staphylococcus aureus ATCC 6538, Candida albicans ATCC 10231, and Aspergillus brasiliensis ATCC 16404.

The present Challenge Test was carried out according to method USP 61 with a cellulose-based composition in accordance with the invention. The composition was prepared by ball milling, and it comprised 5% w/w cellulose and water [Batch: CMC B2AC, 450 G].

The different microbial strains were suspended in a physiologic solution and inoculated in the tested product at a final concentration ranging between 10⁵-10⁶ for all the strains. The treated samples were then stored at room temperature protected from light until plated for the microbial count. The results are shown in Table 9 below.

TABLE 9
Challenge Test of a cellulose-based composition of the invention

| STRAIN | E. coli | P. aeruginosa | S. aureus | C. albicans | A. brasiliensis |
|---|---|---|---|---|---|
| CFU inoculum | 2.2 × 10⁵ | 5.2 × 10⁵ | 7.7 × 10⁵ | 4.5 × 10⁵ | 2.4 × 10⁵ |
| CFU 7 days: | <10 | <10 | <10 | 3.1 × 10⁵ | 4.9 × 10⁴ |
| Microbial reduction (log) | >4.34 | >4.72 | >4.89 | 0.16 | 0.69 |
| Reduction effectiveness | >99.99 | >99.99 | >99.99 | 31.11 | 79.58 |
| (%) | | | | | |
| CFU 14 days: | <10 | <10 | <10 | 2.2 × 10⁵ | 5.1 × 10⁴ |
| Microbial reduction (log) | >4.34 | >4.72 | >4.89 | 0.31 | 0.67 |
| Reduction effectiveness | >99.99 | >99.99 | >99.99 | 51.11 | 78.75 |
| (%) | | | | | |
| CFU 28 days: | <10 | <10 | <10 | 9.0 × 10³ | 6.1 × 10⁴ |
| Microbial reduction (log) | >4.34 | >4.72 | >4.89 | 1.70 | 0.59 |
| Reduction effectiveness | >99.99 | >99.99 | >99.99 | 98.00 | 74.58 |
| (%) | | | | | |

The results of the study were quite positive since the tested composition was effective in microbial reduction (>2 log) for all the stains at 14 days. The tested composition also effectively prevented increase of growth, as measured at 28 days, for all the tested strains. Accordingly, the authors of the study concluded that the cellulose-based composition in accordance with the present invention fully satisfies the requirements of the preservation efficacy test for topically used products, according to USP regulation.

### Example 12: Successful Challenge Testing for Preservation

A USP 51 preservative challenge was conducted with a chitosan-based composition in accordance with the invention [Batch CMCB3.1], the composition further comprising commercially available conservative system Spectrastat™ (Univar Solutions).

Chitosan was pre-milled in isopropanol alcohol using a basket mill BM-100™ for 2.5 hours (mixing speed of 15 hz, milling speed 40/1600 hz, zirconia beads 1.4-1.7 mm). The resulting product was then wet milled using a 1.5 L Supermill PLUS™ horizontal mill. Six (6) passes were conducted at a flow rate of 5.5 Gallons per hour at a mill speed of 2400 FPM, PSI 1-2 using 1.25 mm zirconia beads. Spectrastat™ was next added to the milled product and mixed a few minutes in accordance to the supplier recommendations.

The test was conducted by a Health Canada accredited third party laboratory. Briefly, the test was carried out according to the USP 51 test method by exposing the composition to the following pathogens: A. brasiliensis ATCC 16404, C. albicans ATCC 10231, E. coli ATCC 8739, P. aeruginosa ATCC 9027 and S. aureus ATCC 6538. The composition was examined at three different time intervals (Day 0, Day 14, Day 28) to determine growth of the pathogens. Interestingly, no significant growth in bacteria was found through Day 14 to Day 28 and the authors of the study concluded that “the submitted formula is correctly preserved”.

These results confirm that cosmetic biopolymer compositions according to the present invention may successfully be preserved and that such compositions can prevent growth of pathogens, particularly when the compositions comprise a cosmetically acceptable preservative such as Spectrastat™.

### Example 13: Skin Barrier Effect and Water Resistance Evaluation—Cellulose/Chitosan/Beeswax-Based Composition

In this study, the objective was to demonstrate the waterproof effect of a cellulose/chitosan/beeswax-based composition in accordance with the invention. The cellulose/chitosan/beeswax-based-based composition comprised 5.7% w/w mushroom chitosan, 0.43% w/w alpha cellulose, 5% w/w Dermasoft™, 0.55% cetyl alcohol, and 0.93% beeswax [Batch DEF23051502].

The composition was prepared as follow. First, an original batch “milled formula” comprising alpha cellulose, chitosan and water was milled in a NETZSCH LabStar Zeta Small Media Mill™ for 110 minutes (PSI of 6.9-11, agitator speed 3000 rpm, chiller turned on to maintain temperature of 32° C.). Separately, the following “oily mixture” mixture was made by melting together at 57° C. cetyl alcohol, beeswax synthetic pellets and Dermosoft™. The oily mixture was mixed until the temperature reduced to 52° C. The milled formula (total of 385 grams) was added ⅙^{th} at a time to the oily mixture. Both were mixed together until the temperature went down to 45° C. Once homogenously mixed, this resulting mixture was added very slowly into the ball miller with the remaining of the original batch of milled formula at the 110 min mark. Milling was then continued for 10 further minutes.

The evaluation involved thirteen (13) volunteers and two specialized technicians. The evaluation process was in accordance with Example 10 above.

In the present test, the two specialized technicians gave the tested product a mean score of 9.7 out of 10, for persistence. This very high score indicates a high level of waterproof effect. Accordingly, biopolymer compositions in accordance with the present invention can withstand water splashing after 10 applications.

The study further concluded that the tested product provided “a significant decrease in the Transepidermal water loss (TEWL) values on the skin where the product was applied.” These results further confirm that biopolymer compositions in accordance with the present invention have an important skin barrier effect.

### Example 14: Cellulose/Chitosan Patch Test

A test was carried out by an independent an independent third-party laboratory to assess local skin tolerance of a cellulose biopolymer composition in accordance with the present invention. The composition was of the same batch than Example 7 (i.e., 5% w/w mushroom chitosan, 0.4% w/w cellulose, 5% w/w Dermosoft™ and water) [Batch DEF23041801]. The testing procedure was the same as for the cellulose patch test of Example 9.

In the present test, the Mean Irritation Index (MII) of the test product was 0 at both time periods (15 min and 24 h after patch removal). As such, the test product was classified as “not-irritant” and “dermatologically tested for skin compatibility”, according to the reference standard (data not shown). These results further confirm that cosmetic biopolymer compositions according to the present invention are dermatologically safe.

### Example 15: Reduction of Wrinkles and Fine Lines—Cellulose/Chitosan/Beeswax-Based Composition

In this study, the objective was to evaluate reduction of wrinkles and fine lines reduction using a cellulose/chitosan/beeswax-based composition in accordance with the invention. The composition comprised 5.7% w/w mushroom chitosan, 0.43% w/w alpha cellulose, 5% w/w Dermasoft™, 0.55% cetyl alcohol, and 0.93% beeswax and it was manufactured in accordance with Example 13 above [Batch DEF23051502].

The above composition was evaluated for efficacy by an independent third-party laboratory on 13 healthy male and female volunteers using the same evaluation process as described for Example 7.

The results were very positive in improving crow's feet roughness and wrinkles. Indeed, the study observed a statistically significant reduction of winkles upon application of the tested cellulose/chitosan-based composition. The mean decrease was 5.9% was after only 15 min.

Furthermore, the composition was also useful in “smoothing” the skin. The mean Ra parameter (Linear Average Profile Roughness) demonstrated a decrease by an average value equal to −4.5% after 15 minutes of product application. After 10 days of usage, with an average value equal to −2.2%. The decrease was considered statistically significant for the 15 min time point with respect to the mean value measured before application.

Accordingly, these results clearly demonstrate that biopolymer compositions in accordance with the present invention are active and useful in reducing both wrinkles and fine lines.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a biopolymer” includes one or more of such biopolymers and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A cosmetic composition comprising biopolymer molecules that have been mechanically processed into a stable homogeneous aqueous suspension, and wherein said stable homogeneous aqueous suspension comprises insoluble and/or semi-soluble biopolymer particles stably dispersed within a polar solvent.

2. (canceled)

3. The cosmetic composition according to claim 1, wherein said cosmetic composition consists of a skin care composition which further comprises at least one of a humectant, an emollient, an occlusive, a ceramide, an emulsifier, a thickening agent, a preservative, an antioxidant, a fragrance, and a functional ingredient.

4-27. (canceled)

28. A skin care composition comprising: a biopolymer and at least one of a humectant, an emollient, an occlusive, a ceramide, an emulsifier, a thickening agent, a preservative, an antioxidant, a fragrance, and a functional ingredient.

29. The skin care composition according to claim 28, wherein the composition comprises biopolymer molecules that have been mechanically processed into a stable homogeneous aqueous suspension.

30. The skin care composition according to claim 28, wherein the composition comprises a homogeneous aqueous suspension of insoluble and/or semi-soluble biopolymer particles.

31. The skin care composition according to claim 30, wherein the stable homogeneous aqueous suspension comprises insoluble and/or semi-soluble biopolymer particles stably dispersed within a polar solvent.

32. The skin care composition according to claim 28, wherein the biopolymer consists of a stable homogeneous aqueous suspension composed of insoluble and/or semi-soluble biopolymer particles.

33. The skin care composition according to claim 28, wherein the biopolymer is selected from the group consisting of chitin, chitosan, cellulose, hemicellulose, lignin, amylose, actin, fibrin, collagen, silk, fibroin, keratin, wool, alginic acid and mixtures thereof.

34. (canceled)

35. The skin care composition according to claim 28, wherein the humectant is selected from the group consisting of glycerin, urea, hyaluronic acid, salicylic acid, alpha hydroxy acid, propylene glycol, honey, sorbitol, butylene glycol, sodium PCA, allantoin, sodium lactate, dicyanamide, tremella extract, seaweed, aloe vera and mixtures thereof.

36. The skin care composition according to claim 28, wherein the emollient is selected from the group consisting of petrolatum, lanolin, mineral oil, dimethicone and mixtures thereof.

37. The skin care composition according to claim 28, wherein the occlusive is selected from the group consisting of mineral oil, petrolatum, lanolin, dimethicone, shea butter and mixtures thereof.

38. The skin care composition according to claim 28, wherein the ceramide is selected from the group consisting of ceramide EOS, ceramide NS, ceramide NG, ceramide NP, ceramide AP, ceramide EOP, phytosphingosine, sphingosine and mixtures thereof.

39. The skin care composition according to claim 28, wherein the emulsifier is selected from the group consisting of sodium lauryl sulfate, sodium dioctyl sulfosuccinate, tragacanthins, lecithin, mustard, soy lecithin, sodium phosphates, diacetyl tartaric acid ester of monoglyceride, sodium stearoyl lactylate, and mixtures thereof.

40. The skin care composition according to claim 28, wherein the thickening agent is selected from the group consisting of magnesium aluminium, silicate, silica, bentonite, carbomer, cetyl palmitate, ammonium acryloyldimethyltaurate and mixtures thereof.

41. The skin care composition according to claim 28, wherein the preservative is selected from the group consisting of phenoxyethanol, benzyl alcohol, sodium benzoate, potassium sorbate, ethylhexylglycerin, DMDM hydantoin, quaternium-15, imidazolidinyl urea, diazolidinyl urea, polyoxymethylene urea, sodium hydroxymethylglycinate, 2-bromo-2-nitropropane-1,3-diol (bromopol), glyoxal, ethylparaben, butylparaben, methylparaben, propylparaben, isobutylparaben, isopropylparaben, benzylate, benzoic acid, benzyl ester, methylisothiazolinone (MIT; 2-methyl-4-isothiazoline-3-one), methylchloroisothiazolinone (CMIT; 5-chloro-2-methyl-4-isothiazolin-3-one), phenoxyethanol, 2-phenoxyethanol, Euxyl K™400 (mixture of phenoxyethanol and 1,2-dibromo-2,4-dicyanobutane), triclosan (TSC), triclocarban (tcc), benzyl alcohol, benzalkonium chloride, citric acid, dehydroacetic acid, essential oils, grapefruit seed extract, lactic acid, levulinic acid, potassium sorbate, sodium dehydroacetate, sodium metabisulfite, sodium salicylate, sorbic acid, vitamin E, zinc pyrithione and mixtures thereof.

42. The skin care composition according to claim 28, wherein the antioxidant is selected from the group consisting of vitamin A, vitamin C, vitamin E, vitamin B3, green tea extract, resveratrol, curcumin, lycopene, coenzyme Q10, polyphenols, C. asiatica, tamanu, and mixtures thereof.

43. The skin care composition according to claim 28, wherein the fragrance is selected from the group consisting of linalool, citronellol, limonene, geraniol, eugenol, lavender oil (Lavandula angustifolia), rose flower extract (Rosa damascena), bergamot oil (Citrus bergamia), ylang-ylang oil (Canaga odorata), lemon (Citrus limon), lime (Citrus aurantifolia or Citrus medica), orange (Citrus sinensis), tangerine (Citrus tangerine), peppermint (Mentha piperita), spearmint (Mentha spicata), eucalyptus, cinnamon (Cinnamomum)/, cinnamal and mixtures thereof.

44. The skin care composition according to claim 28, wherein the functional ingredient is selected from the group consisting of biological extracts, vitamins, amino acids, peptides, proteins, essential fatty acids, hydroxy acids, beta-glucans and mixtures thereof.

45. The skin care composition according to claim 28, wherein said skin care composition is formulated as a paste, an ointment, a cream, a lotion, a gel and/or a milk.

46. (canceled)

47. (canceled)

48. A method of protecting the skin and/or improving appearance of the skin, comprising applying onto the skin a skin care composition as defined in claim 28.

49-67. (canceled)
